(19) United States
(12) Patent Application Publication
(10) Pub. No.: US 2008/0293070 A1
(43) Pub. Date: Nov. 27, 2008

(54) Markers for Memory T Cells and Uses Thereof

(76) Inventors: Rafick-Pierre Sekaly, Saint-Laurent (CA); Elias Haddad, Saint-Laurent (CA); Catherine Riou, Montreal (CA)

Correspondence Address: WELLS ST. JOHN P.S., 601 W. FIRST AVENUE, SUITE 1300, SPOKANE, WA, 99201, US

(21) Appl. No.: 12/158,201

(22) PCT Filed: Dec. 21, 2006
(86) PCT No.: PCT/CA2006/002103

§ 371 Date: Jun. 19, 2008

Related U.S. Application Data
(60) Provisional application No. 60/752,042, filed on Dec. 21, 2005.

(51) Int. Cl. C12Q 1/68; C12Q 1/02; C12N 5/00; C07H 21/00; C40B 40/06

(52) U.S. Cl. 435/6; 435/29; 435/375; 536/23.1; 506/16

(57) ABSTRACT

Methods, uses, products and kits are described relating to monitoring, assessing and modulating immune function and more particularly memory T cell function. Methods of identifying agents for such modulation are also described, as well as uses of such agents for modulating immune function.

### CROSS REFERENCE TO RELATED APPLICATIONS

This application claims the benefit, under 35 U.S.C. § 119(e), of U.S. provisional application Ser. No. 60/752,042 filed on Dec. 21, 2005, which is incorporated herein by reference in its entirety.

### FIELD OF THE INVENTION

The present invention relates to immune memory. More specifically, the present invention is concerned with reagents and methods for monitoring and modulating the immune response and memory T cells.

### BACKGROUND OF THE INVENTION

The generation and maintenance of memory T cells is central to the development of protective immunity, as characterized by a rapid and vigorous response following the encounter with a given pathogen or antigen (Kaech, S. M et al., Nat Rev Immunol 2:251-262; Sallusto, F et al., Annu Rev Immunol 22:745-763). Despite the complexity of the memory T cell populations, recent studies in both mice and humans indicate that the memory T cell pool is composed of two main compartments, central memory T cells (T_{CM}) and effector memory T cells (T_{EM}), which are characterized by distinct homing capacities and effector functions (Sallusto, F. et al., Nature 401:708-712; Fritsch, R. D. et al., J Immunol 175:6489-6497). Through their expression of CCR7 and CD62L, T_{CM} preferentially home to T-cell areas of secondary lymphoid organs and display little immediate effector functions; however, they readily proliferate and differentiate to effector cells in response to antigenic stimulation. T_{EM}, which have lost the constitutive expression of CCR7, express tissue homing receptors associated with inflammation and display more readily-effector functions.

The current model proposes that upon re-infection, T_{EM} rapidly constrain pathogen invasion in inflamed peripheral tissues, whereas T_{CM} are rapidly activated by dendritic cells (DCs) in secondary lymphoid organs and generate successive waves of effectors able to completely eliminate the pathogen (Sallusto, F et al., Annu Rev Immunol 22:745-763). Experiments performed in murine models suggest that T_{CM} have a better capacity to reconstitute the memory T-cell pool and to mediate protective immunity than T_{EM}, due to their greater capacity to proliferate and persist in vivo (Wu, C. Y. et al., Nat Immunol 3:852-858, Zaph, C. et al., Nat Med 10:1104-1110). Studies in primate models show that induction of central memory CD4⁺ T cells following SIV challenge correlates with prolonged survival (Letvin, N. L. et al., Science 312:1530-1533), thereby highlighting the importance of gaining a better understanding of the mechanisms underlying T_{CM} induction and persistence for successful vaccine development. The long-term maintenance of memory T cells relies on the survival of individual cells and their level of homeostatic cell division to compensate for their gradual attrition through apoptosis (Sallusto, F et al., Annu Rev Immunol 22:745-763; Sad, S., and L. Krishnan, Crit Rev Immunol 23:129-147). Using in vivo labeling with deuterated glucose to measure the turnover of distinct subsets of CD4⁺ T cells in healthy humans, Macallan et al. have shown that T_{EM} have a more rapid turnover than T_{CM}, suggesting that T_{EM} are being replaced at a faster rate than T_{CM} (Macallan, D. C. et al., J Exp Med 200:255-260). Studies in mouse models have suggested that signaling through TCR and γ-chain cytokine receptors might play a role for long-term survival of memory T cells (Seddon, B. et al., Nat Immunol 4:680-686; Kondrack, R. M. et al., J Exp Med 198:1797-1806; Patke, D. S., and D. L. Farber. J Immunol 174:5433-5443; Kassiotis, G. et al., J Exp Med 197:1007-1016). For example, memory CD4 cells persisted for extended periods upon adoptive transfer into intact or lymphopenic recipients but not in IL-7^{-/-} mice (Kondrack, R. M. et al., J Exp Med 198:1797-1806). Moreover, Kassiotis et al. have demonstrated that the homeostatic expansion capacity of both CD4⁺ naïve and memory cells is dependent upon the expression levels of TCR and CD5, a negative regulator of TCR signaling (Kassiotis, G. et al., J Exp Med 197:1007-1016).

Given the importance of memory T cells, and particularly central memory T cells, in the protection from various diseases such as infectious diseases, there is a need to develop new reagents and methods that influences their induction/maintenance and that permits their identification/detection.

The present description refers to a number of documents, the content of which is herein incorporated by reference in their entirety.

### SUMMARY OF THE INVENTION

The invention relates to methods, products, uses and kits for monitoring and modulating the immune response and memory T cells.

The present invention provides a method of identifying an agent capable of (a) inducing the level of memory T cells, (b) promoting the survival of memory T cells, or (c) both (a) and (b), comprising determining Foxo3a phosphorylation in the presence versus the absence of a test agent, wherein a higher level of phosphorylated Foxo3a in the presence of the agent is indicative that the agent is capable of (a) inducing the level of memory T cells, (b) promoting the survival of memory T cells, or (c) both (a) and (b).

In an embodiment, the above-mentioned phosphorylation is at a Foxo3a residue corresponding to Thr32, Ser253, Ser315, or any combination thereof.

In an embodiment, the above-mentioned memory T cell is a central memory T cell (T_{CM}).

In an other aspect, the present invention provides a method of identifying an agent capable of (a) inducing the level of memory T cells, (b) promoting the survival of memory T cells, or (c) both (a) and (b), comprising determining the expression of one or more nucleic acids or polypeptides comprising a sequence selected from SEQ ID NOs: 10-201 in a biological sample from an animal prior to versus after contacting the sample with a test agent, wherein a modulation of expression after contact with the agent relative to prior to contact with the agent is indicative that the agent is capable of (a) inducing the level of memory T cells, (b) promoting the survival of memory T cells, or (c) both (a) and (b).

In an embodiment, the above-mentioned memory T cells are central memory T cells, the above-mentioned modulation is an increase and the above-mentioned one or more nucleic acids or polypeptides comprises a sequence selected from SEQ ID NOs: 10-125 and 198-199.

In another embodiment, the above-mentioned memory T cells are effector memory T cells, the above-mentioned modulation is an increase and the above-mentioned one or more nucleic acids or polypeptides comprises a sequence selected from SEQ ID NOs: 126-197 and 200-201.

In an embodiment, the level of expression of at least 2 nucleic acids or polypeptides is determined. In an embodiment, the level of expression of at least 5 nucleic acids or polypeptides is determined. In an embodiment, the level of expression of at least 10 nucleic acids or polypeptides is determined. In an embodiment, the level of expression of at least 25 nucleic acids or polypeptides is determined. In an embodiment, the level of expression of at least 50 nucleic acids or polypeptides is determined.

In an embodiment, the above-mentioned one or more nucleic acids or polypeptides comprises a sequence selected from SEQ ID NOs: 12-25, 38-39, 50-53, 62-63, 82-83, 92-95, 100-107, 110-113, 126-129, 140-151, 154-169 and 174-187.

In an embodiment, the above-mentioned one or more nucleic acids or polypeptides comprises a sequence selected from SEQ ID NOs: 12-25, 38-39, 50-53, 62-63, 82-83, 92-95, 100-107 and 110-113.

In an embodiment, the above-mentioned one or more nucleic acids or polypeptides comprises a sequence selected from SEQ ID NOs: 126-129, 140-151, 154-169 and 174-187.

In an embodiment, the above-mentioned method further comprises determining the expression of one or more genes or polypeptides encoded thereby set forth in FIG. 2B.

In an other aspect, the present invention provides a method of identifying an agent capable of inducing protective immunity in an animal, comprising:
(i) providing a first expression profile of one or more nucleic acids or encoding polypeptides selected from BIRC5, CALM1, CAMK2G, CaMKIINalpha, DC-UbP, FAIM2, FOXL2, GATA2, GATA3, IL-7R, IRF1, KIT, MAPK6, MAPKAPK3, RAB11B, STMN1, TNFRSF7 (CD27), CLK1 and PRKARI B in a biological sample from an animal prior to contacting the sample with a test agent;
(ii) providing a second expression profile of one or more nucleic acids encoding a polypeptide selected from BIRC5, CALM1, CAMK2G, CaMKIINalpha, DC-UbP, FAIM2, FOXL2, GATA2, GATA3, IL-7R, IRF1, KIT, MAPK6, MAPKAPK3, RAB11B, STMN1, TNFRSF7 (CD27), CLK1 and PRKARI B in a biological sample from an animal after contacting the sample with the test agent;
(iii) providing a reference expression profile associated with the expression of one or more nucleic acids encoding a polypeptide selected from BIRC5, CALM1, CAMK2G, CaMKIINalpha, DC-UbP, FAIM2, FOXL2, GATA2, GATA3, IL-7R, IRF1, KIT, MAPK6, MAPKAPK3, RAB11B, STMN1, TNFRSF7 (CD27), CLK1 and PRKARI B in a biological sample from an animal exhibiting protective immunity;
   wherein increased similarity of the second expression profile to the reference expression profile, relative to the first expression profile to the reference expression profile, is indicative that the agent is capable of inducing protective immunity.

In an other aspect, the present invention provides a method of identifying an agent capable of inducing protective immunity in an animal, comprising determining the expression of one or more nucleic acids or polypeptides selected from BIRC5, CALM1, CAMK2G, CaMKIINalpha, DC-UbP, FAIM2, FOXL2, GATA2, GATA3, IL-7R, IRF1, KIT, MAPK6, MAPKAPK3, RAB11B, STMN1, TNFRSF7 (CD27), CLK1 and PRKARI B in a biological sample from an animal prior to versus after contacting the sample with a test agent, wherein a modulation of expression after contact with the agent relative to prior to contact with the agent is indicative that the agent is capable of inducing protective immunity.

In an embodiment, the above-mentioned modulation is an increase and the above-mentioned one or more nucleic acids or polypeptides is selected from BIRC5, CALM1, CAMK2G, CaMKIINalpha, DC-UbP, FAIM2, FOXL2, GATA2, GATA3, IL-7R, IRF1, KIT, MAPK6, MAPKAPK3, RAB11B, STMN1 and TNFRSF7 (CD27).

In an other embodiment, the above-mentioned modulation is a decrease and the above-mentioned one or more nucleic acids or encoding polypeptides is selected from CLK1 and PRKARI B.

In an embodiment, the above-mentioned agent is a vaccine.

In an embodiment, the above-mentioned subject exhibiting protective immunity is a subject vaccinated with a vaccine known to confer immune protection. In a further embodiment, the above-mentioned vaccine is Yellow Fever vaccine.

In an embodiment, the above-mentioned method comprises providing the expression profile of at least 2 nucleic acids or polypeptides.

In an embodiment, the above-mentioned method comprises providing the expression profile of at least 5 nucleic acids or polypeptides.

In an embodiment, the above-mentioned method comprises providing the expression profile of at least 10 nucleic acids or polypeptides.

In an embodiment, the above-mentioned biological sample is a tissue or body fluid. In a further embodiment, the above-mentioned biological sample is blood or comprises a blood cell. In a further embodiment, the above-mentioned blood cell is a Peripheral Blood Mononuclear Cell (PBMC). In a further embodiment, the above-mentioned Peripheral Blood Mononuclear Cell (PBMC) is an immune cell. In a further embodiment, the above-mentioned immune cell is a CD4⁺ or CD8⁺ memory T cell. In a further embodiment the above-mentioned memory T cell is a central memory T cell (T_{CM}).

In an embodiment, the above-mentioned level of expression or expression profile is determined at the nucleic acid level using a technique selected from the group consisting of Northern blot analysis, reverse transcription PCR, real time quantitative PCR, microarray analysis and RNase protection.

In an other embodiment, the above-mentioned level of expression or expression profile is determined at the polypeptide level. In an other embodiment, the above-mentioned level of expression or expression profile of the polypeptide is determined using a reagent which specifically binds with the polypeptide. In a further embodiment, the above-mentioned reagent is an antibody or an antigen binding fragment thereof.

In an other embodiment, the above-mentioned level of expression or expression profile is determined using a method selected from the group consisting of Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), flow cytometry and antibody microarray.

In an other aspect, the present invention provides a method of inducing the survival of a memory T cell, said method comprising contacting said cell with an agent capable of phosphorylating Foxo3a.

In an other aspect, the present invention provides a method of increasing immune function in a subject, said method comprising inducing the phosphorylation of Foxo3a in an immune cell of said subject.

In an embodiment, the above-mentioned immune function is memory T cell function. In a further embodiment, the above-mentioned memory T cell function is memory T cell survival or persistence.

In an other aspect, the present invention provides a method of determining whether an HIV-positive subject possesses natural resistance to the development of AIDS, said method comprising:
(i) providing a first expression profile of one or more nucleic acids encoding a polypeptide selected from XIAP, GADD45, DUSP1, PTEN, SOCS1 and SOCS2 in a biological sample from said subject,
(ii) providing a reference expression profile of said one or more nucleic acids in a biological sample from a reference subject known to be an HIV-positive long term non-progressor,
   wherein a similarity of the first expression profile to the reference expression profile is indicative that the HIV-infected subject possesses natural resistance to the development of AIDS

In an other aspect, the present invention provides a collection of two or more isolated nucleic acid sequences which are substantially identical to two or more isolated respective nucleic acid sequences encoding two or more respective polypeptides selected from SEQ ID NOs: 10-201, their complements or portions thereof.

In an embodiment, the above-mentioned collection comprises at least 5 isolated nucleic acid sequences encoding at least 5 polypeptides, their complements or portions thereof.

In an embodiment, the above-mentioned collection comprises at least 10 isolated nucleic acid sequences encoding at least 10 polypeptides, their complements or portions thereof.

In an embodiment, the above-mentioned collection comprises at least 25 isolated nucleic acid sequences encoding at least 25 polypeptides, their complements or portions thereof.

In an embodiment, the above-mentioned collection comprises at least 50 isolated nucleic acid sequences encoding at least 50 polypeptides, their complements or portions thereof.

In an embodiment, the above-mentioned collection comprises isolated nucleic acid sequences encoding all polypeptides defined above, their complements or portions thereof.

In an embodiment, the above-mentioned isolated nucleic acid sequences are immobilized on a substrate.

In an embodiment, the above-mentioned isolated nucleic acid sequences are conjugated to a detectable marker.

In an embodiment, the above-mentioned isolated nucleic acid sequences are hybridizable array elements in a microarray.

In an other aspect, the present invention provides an array comprising a substrate and a collection of bound nucleic acids, each of said nucleic acids being bound to said substrate at a discrete location, wherein said collection of bound nucleic acids is the collection defined above.

In an other aspect, the present invention provides a composition for the prevention or treatment of an immune disease in a subject, said composition comprising:
(i) an agent capable of (a) phosphorylating Foxo3a in an immune cell, (b) increasing the expression of one or more nucleic acids or encoding polypeptides comprising a sequence selected from SEQ ID NOs: 12-25, 38-39, 50-53, 62-63, 82-83, 92-95, 100-107 and 110-113, (c) both (a) and (b), in said subject; and
(ii) a pharmaceutically acceptable carrier.

In an other aspect, the present invention provides a use of the above-mentioned composition for the prevention or treatment of an immune disease.

In an other aspect, the present invention provides a use of the above-mentioned composition for the preparation of a medicament for the prevention or treatment of an immune disease.

In an other aspect, the present invention provides a use of an agent capable of (a) phosphorylating Foxo3a, (b) increasing the expression of one or more nucleic acids or encoding polypeptides selected from comprising a sequence selected from SEQ ID NOs: 12-25, 38-39, 50-53, 62-63, 82-83, 92-95, 100-107 and 110-113, (c) both (a) and (b) for the prevention or treatment of an immune disease.

In an other aspect, the present invention provides a use of an agent capable of (a) phosphorylating Foxo3a, (b) increasing the expression of one or more nucleic acids or encoding polypeptides comprising a sequence selected from SEQ ID NOs: 12-25, 38-39, 50-53, 62-63, 82-83, 92-95, 100-107 and 110-113, (c) both (a) and (b) for the preparation of a medicament for the prevention or treatment of an immune disease.

In an other aspect, the present invention provides a package comprising the above-mentioned composition together with instructions for its use for the prevention or treatment of an immune disease.

In an other aspect, the present invention provides a package comprising:
(i) an agent capable of (a) phosphorylating Foxo3a, (b) increasing the expression of one or more nucleic acids or encoding polypeptides comprising a sequence selected from SEQ ID NOs: 12-25, 38-39, 50-53, 62-63, 82-83, 92-95, 100-107 and 110-113, (c) both (a) and (b) in a subject; and
(ii) instructions for its use for the treatment or prevention of an immune disease in said subject

In an embodiment, the above-mentioned immune disease is immune deficiency. In a further embodiment, the above-mentioned immune deficiency is a deficiency in a memory T cell. In a further embodiment, the above-mentioned memory T cell is a central memory T cells (T_{CM}). In a further embodiment, the above-mentioned central memory T cells (T_{CM}) is a CD4⁺ central memory T cell.

In an embodiment, the herein-mentioned nucleic acid, polypeptide or gene is associated with apoptosis and/or cell survival including any pathway related thereto. Examples of such genes are set forth in FIG. 3.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

FIG. 1: Functional and phenotypical characterization of CD4+T_{CM} and T_{EM}-(A) CD45RA, CD27 and CCR7 labeling profile and gating strategy for naïve, T_{CM} and T_{EM}. Percentages obtained for each population are indicated. Purity of sorted cells was always above 95%. (B) Perforin and Granzyme-B (Gr-B) expression in ex-vivo T_{CM} and T_{EM} subsets. Perforin and Gr-B expression were assayed by intracellular staining. The percentages of T_{CM} and T_{EM} expressing Perforin and Gr-B are indicated in each quadrant. (C) Rab27a protein levels in ex-vivo sorted T_{CM} and T_{EM} subsets. Similar results were obtained in three independent experiments. (D) Susceptibility of T_{CM} and T_{EM} towards Fas-induced apoptosis. T_{CM} and T_{EM} were sorted by flow cytometry and treated with anti-Fas antibodies (CH11:1.25 μg/ml) or etoposide (100 μg/ml) for 24 hours. The percentage of apoptotic cells was assessed by flow cytometry using Annexin-V labeling. The results are depicted as a percentage of apoptotic cells ±SD of three independent experiments. (E) Proliferation and persistence of purified T_{CM} and T_{EM}. Sorted T_{CM} and T_{EM} were co-cultured with autologous mature dendritic cells in the presence of superantigen (staphylococcal enterotoxin A, SEA) for 15 days. After 1 to 15 days, the proportion of proliferating cells was assessed by staining of anti-TCRVβ22, as Vβ22 is known to be a highly SEA-reactive Vβ (Lavoie P M et al., 2005. Int Immunol., 17(7):931-41). Results are represented as the % of Vβ22 positive cells.

FIG. 2. Gene expression profiling of CD4⁺ T_{CM} and T_{EM}. CD4⁺ T cell subsets (central and effector memory) were purified from blood samples collected from healthy individuals by flow cytometry-based cell sorting using monoclonal antibodies directed against CD4, CD45RA, CCR7 and CD27. Messenger RNA (mRNA) was isolated from sorted CD4⁺ T_{CM} and T_{EM}, converted into cDNA and gene expression was analyzed by cDNA microarray. A. List of most significant genes differentially expressed between T_{CM} and T_{EM}. B. List of other genes whose expression differs between T_{CM} and T_{EM}. Genes expressed at higher level in T_{EM} as compared to T_{CM} are highlighted in grey. Each gene on the arrays was performed in duplicate to avoid false-positive signals. Fold change values were obtained from the average value of 13 independent hybridizations. The p-values were determined by ANOVA, based on F-test. Avg FDE=Average fold difference in expression in T_{CM} vs. T_{EM} (positive numbers represent genes having a higher expression in T_{CM} whereas negative numbers represent genes having a higher expression in T_{EM}.

FIG. 3. Differential expression of apoptosis-related genes in CD4⁺ T_{CM} and T_{EM}. Significant genes were selected using ANOVA t-test (p<0.05 or fold change >1.3) and associated with an “apoptosis” GO annotation. Each gene on the arrays was spotted in duplicate to avoid false-positive signals and to ensure the reproducibility of the data obtained. The fold-change values were obtained from the average value of thirteen independent hybridizations (AVG FC). The genes upregulated in T_{EM} are highlighted in grey. The p-values were determined by ANOVA, based on F-test. Fold change values were calculated from the average value of thirteen independent hybridizations by subtracting the mean expression of the log 2 ratio obtained in T_{CM} from the log 2 ratio obtained in T_{EM}. That value was then converted into fold change.

FIG. 4. Quantification of gene expression in CD4⁺ T_{CM} and T_{EM} by RT-PCR. CD4 T cell subsets (central and effector memory) were purified as described above. Messenger RNA (mRNA) was isolated from sorted CD4⁺ T_{CM} and T_{EM}, converted into cDNA, and the expression of selected genes (listed in the first column) was analyzed by quantitative RT-PCR using primers specific for each gene. The primers were synthesized by Applied Biosystems based on the following context sequences:

| | |
|---|---|
| CDKN1B (p27kip): | |
| AACCGACGATTCTTCTACTCAAAAC; | (SEQ ID NO: 1) |
| CD27: | |
| GCACTGTAACTCTGGTCTTCTCGTT; | (SEQ ID NO: 2) |
| GADD45a: | |
| TGCGTGCTGGTGACGAATCCACATT; | (SEQ ID NO: 3) |
| DUSP6: | |
| CCATTTCTTTCATAGATGAAGCCCG; | (SEQ ID NO: 4) |
| PIM2: | |
| TCCCCCTTGTCAGACTCAGTCACAT; | (SEQ ID NO: 5) |
| pRb2/p130: | |
| ATTTTGACAAGTCCAAAGCACTTAG; | (SEQ ID NO: 6) |
| FasL: | |
| GAAGCAAATAGGCCACCCCAGTCCA; | (SEQ ID NO: 7) |
| Bim: | |
| TCAGTGCAATGGCTTCCATGAGGCA; | (SEQ ID NO: 8) |
| LKLF: | |
| CTGCAGGAGCGCTGGCCGCGCGCCG | (SEQ ID NO: 9) |

The numbers indicate the fold up-regulation of transcript level in T_{CM} vs. T_{EM} (second column) or T_{EM} vs. T_{CM} (third column), following normalization to GAPDH and Actin levels. These results represent the average value of two independent experiments performed on sample from two different blood donors.

FIG. 5. STAT5a signaling pathway is functionally upregulated in T_{CM}. (A) PIM-1 and PIM-2 proteins levels in ex-vivo sorted T_{CM} and T_{EM} subsets. Similar results were obtained in three independent experiments. (B, C and D) PBMCs from healthy donors were treated with IL-2 (100 U/ml) or IL-7 (10 ng/ml) for 15 min at 37° C. Cells were then labeled with Abs to CD4, CD27, CCR7, CD45RA and pSTAT5a. (B) Representative example of pSTAT5a expression levels. T_{CM}-gated cells are represented in light grey and T_{EM}-gated cells are represented in black. (C) Mean Fluorescence Intensity (MFI) of pSTAT5a level of expression measured in response to IL-2 or IL-7, in T_{CM} and T_{EM} (n=4). Mean pSTAT5a signal values are represented by black bars. P-values (determined by a two-tailed T-test) are shown (D) Expression level of CD127, CD25 and CD132 in ex-vivo T_{CM} and T_{EM}. PBMCs were labeled with Abs to CD4, CD27, CCR7 and CD45RA to identify T cells subsets in conjunction with anti-CD127 or anti-CD25 specific Abs. The results represent the proportions of CD127 and CD25 positive cells on T_{CM}- and T_{EM}-gated T cells (% of positive cells ±SD of five independent experiments).

FIG. 6. Regulation of the FOXO3a pathway in memory CD4⁺ T cell subsets. (A) FOXO3a, pFOXO3a (S315, S253 or T32), Bim, p130 and Gadd45a protein levels in ex-vivo sorted T_{CM} and T_{EM}. (B) Expression of FasL on activated T_{CM} and T_{EM}. PBMC were activated with phorbol myristate acetate (PMA) (10 ng/ml) and lonomycin (500 ng/ml) for 24 hours. Intracellular staining was performed using CD4, CD27, CD45RA, CCR7 and FasL antibodies. The percentages of FasL positive cells for each subset are indicated. MFI values are indicated in brackets.

FIG. 7. AKT and IKK mediate FOXO3a phosphorylation and survival in CD4⁺ T cell. (A) Regulation of FOXO3a phosphorylation. Purified CD4⁺ T Cells were pre-treated for 1 hour with kinase inhibitors (AKT-VI, AKT inhibitor: 10 μM; STO-609, CamKK inhibitor: 5 μg/ml; Wedelolactone, IKK inhibitor: 100 μM and U0126, MEK1/2 inhibitor: 50 μM). pFoxo3a (S253) was assessed. The results are representative of two independent experiments. (B) CD4⁺ T cell susceptibility to apoptosis induced upon treatment with specific kinase inhibitors. CD4⁺ T cells were cultured in the presence of kinase inhibitors for 24 hrs (U0126:100 μM; STO-609:10 μg/ml; Wedelolactone and AKT-IV as indicated). After 24 h, the proportion of Annexin-V⁺, propidium iodide (PI)⁺ cells was quantified by flow cytometry. The results are depicted as a percentage of apoptotic cells within the total population and are representative of two independent experiments. (C) Bim expression levels in response to AKT and IKK inhibitors. CD4⁺ T cells were treated with AKT-IV (1.6 μM) or wedelolactone (100 μM) for 24 h. Cells were analyzed by Western Blot (WB) using Bim specific antibodies. (D and E) Regulation of AKT and IKK phosphorylation in 4⁺ memory subsets. (D) pIKKαβ (S176/180) protein levels in ex-vivo sorted T_{CM} and T_{EM}. Prolonged exposure did not reveal any pIKK signal in T_{EM}. Similar results were obtained in three independent experiments. (E) PBMCs, from healthy donors, were treated with H₂O₂ (5 mM) or Ig-cross-linked CD28 (2 Hg/ml) for 15 min at 37° C. and labeled with CD4, CD27, CD45RA and pAKT (S473) specific antibodies. The levels of pAKT were assessed by flow cytometry in T_{CM}- and T_{EM}-gated subsets. The results are represented as the mean fold increase ±SD, calculated as (MFI of stimulated cells/MFI of un-stimulated cells) of five independent experiments. Values of p (determined by two-tailed T-test) are shown.

FIG. 8. Susceptibility of T_{CM} and T_{EM} to apoptosis induced by kinase inhibitors. Sorted T_{CM} and T_{EM} were cultured with or without AKT and IKK inhibitors as indicated. After 24 h, the percentage of apoptotic cells was quantified by Annexin-V-FITC labeling. Upper panel depict Results from a representative individual. Histogram plots show the percent of Annexin-V positive cells in T_{CM} and T_{EM} cells following a 24 h exposure to AKT or IKK-inhibitors. The dashed lines correspond to untreated cells while the plain lines correspond to cells treated with kinase-inhibitors. Lower panel is a bar graph representation of the fold increase of apoptosis in T_{CM} and T_{EM} in response to IKK or AKT inhibitor. The fold increase of apoptosis is calculated as % of apoptotic cells in treated cells divided by the % of apoptotic cells in untreated cells. Similar results were obtained in two independent experiments.

FIG. 9. FOXO3a phosphorylation is induced by TCR and cytokine engagement. (A) CD4⁺ T cells were cultured in the presence of anti-CD3 (2 μg/ml), anti-CD28 (2 μg/ml), anti-CD3+CD28, IL-2 (100 U/ml), IL-7 (10 ng/ml), IFN-γ (50 μg/ml) or PMA (50 ng/ml) for 15 min and analyzed by Western Blot for pFOXO3a (S315 and S253) expression levels. The results are representative of two independent experiments. (B) CD4⁺ T cells were cultured in the presence of anti-CD3/anti-CD28, IL-2 (100 U/ml), IL-7 (10 ng/ml) for 30 min and analyzed by Western Blot for pFOXO3a (T32) expression levels. Similar results were obtained in two independent experiments.

FIG. 10. Comparison of the expression of selected genes in T_{CM} and T_{EM} isolated from aviremic HAART-treated HIV-infected individuals vs. long-term non-progressors (LTNPs) Cells were sorted from PBMC obtained from LTNP and aviremic HAART-treated patients into T_{CM} and T_{EM} using CD27, CCR7 and CD45RA surface markers. Sorted cells were subjected to RNA isolation, amplification and gene array analysis. This figure shows differences in T_{CM} and T_{EM} from LTNP versus T_{CM} and T_{EM} from aviremic chronically infected HIV patients.

FIG. 11. Comparison of the expression of genes in blood samples isolated from Yellow Fever-vaccinated individuals. Blood samples were collected at different time points (before vaccination (day 0), day 3 and 7 post-vaccination) from 8 subjects vaccinated against Yellow Fever (Yellow Fever 17D vaccine).

### DESCRIPTION OF ILLUSTRATIVE EMBODIMENTS

In the studies described herein, the gene expression profile of subpopulations of memory CD4 T cells was analyzed. It was found that several genes are differentially expressed in central memory T cells (T_{CM}) vs. effector memory T cells (T_{EM}), notably genes associated with cell survival/apoptosis. T_{CM} tend to express higher levels of specific genes associated with cell survival and inhibition of apoptosis, whereas T_{EM} generally express higher levels of genes associated with induction of apoptosis.

Accordingly, the present invention relates to monitoring/detecting as well as modulating memory T cells based on such correlation of gene expression.

The invention provides a screening method for identifying agents/compounds that can be used for (a) induce the level of memory T cells, (b) promote the survival of memory T cells, or (c) both (a) and (b) based on their ability to phosphorylate Foxo3a. In an embodiment, the method comprises determining Foxo3a phosphorylation in the presence versus the absence of the agent. A higher level of phosphorylated Foxo3a in the presence of the agent is indicative that the agent is capable of (a) inducing the level of memory T cells, (b) promoting the survival of memory T cells, or (c) both (a) and (b). In an embodiment, the phosphorylation of Foxo3a is at a Foxo3a residue corresponding to Thr32, Ser253, Ser315, or any combination thereof. The extent of Foxo3a phosphorylation can be determined, for example, using antibodies specific for one or more phosphorylated forms of Foxo3a (see Example 6).

In another embodiment, the method comprises determining the expression of one or more nucleic acids encoding a polypeptide selected from HLA-G, MAL, NGFRAP1, HRMT1L2, ATXN3, TNFRSF7 (CD27), ING1, E2F4, RELA, TOSO, INDO, SFRP4, PABPC1, ARL7, PIM2, TAP1, CD37, LPPR4, IMPDH2, LOC112476, TGFBR2, CCNL1, GRK5, Stat5a, RALA, CSTB, SNF1LK, CAV1, MYO1E, B2M, NFIC, SYT6, RRM1, OAS1, IMPDH2, DMGDH, PNRC2, LIMS1, PARVG, FYN, LILRA2, FTL, SOCS1, PF4, ERG, IFIT1, NCOR2, IL16, TCIRG1, PITPNB, PABPC4, MAN2A1, SPN, TNFRSF8, RFX2, RGS13, LTA4H, S100A8, TCF3, TIAM1, CART, PPP2R2C, PIAS4, PRKCQ, NME2, SLC2A3, ATF4, IL2RG, COL3A1, PPM1D, SEC23A, LIMK2, BAT3, RGS10, STAT6, RASL12, C1QG, GPR18, NOTCH3, C1orf38, BTF3, CCL19, PES1, C1QA, ZNF593, TNF-α, POLD2, DTYMK, E2F1, STAU, IFNGR2, NRG1, TNFSF7, JARIDLA, BLR1, PLCL2, MKI67, IDUA, FEZ1, MAPKAPK5, DLC1, MAP4K2, VAV3, BATF, BIRC6, CEP2, DDAH2, PLK4, GTF2F2, FADS1, FHIT, SPOCK, TLK1, DDX5, NGFR, FYB, USP10, TCF7, RAMP1, AGPAT5, EDA, PPP3CC, HNRPK, TPR, CHUK, ANXA1, SMARCA4, CLK1, CCL3, CALM3, ALOX5, LCN2, NUP88 and LKLF in a biological sample from an animal prior to versus after administration of a test agent/compound to the subject, or in a biological sample from an animal or prior to versus after contacting the sample with a test agent. A higher level of expression following the administration is indicative that the agent is capable of capable of (a) inducing the level of memory T cells, (b) promoting the survival of memory T cells, or (c) both (a) and (b).

In an embodiment, the above-mentioned memory T cells are central memory T cells (T_{CM}). In a further embodiment, the above-mentioned central memory T cells are 4⁺ central memory T cells.

The agents/compounds identified by these screening methods can be used for the prevention or treatment of immune disorders, and more particularly immune deficiencies associated with low levels of memory T cells.

In another aspect, the present invention provides a method of determining whether an agent (e.g., a vaccine or an immunotherapeutic agent) is capable of inducing protective immunity in an animal, comprising:

(i) providing a first expression profile of one or more nucleic acids encoding a polypeptide selected from BIRC5, CALM1, CAMK2G, CaMKIINalpha, DC-UbP, FAIM2, FOXL2, GATA2, GATA3, IL-7R, IRF1, KIT, MAPK6, MAPKAPK3, RAB11B, STMN1, TNFRSF7 (CD27), CLK1 and PRKARI B in a biological sample from said animal prior to administration of the agent to the subject, or in a biological sample from an animal prior to contacting the sample with the agent,

(ii) providing a second expression profile of one or more nucleic acids encoding a polypeptide selected from BIRC5, CALM1, CAMK2G, CaMKIINalpha, DC-UbP, FAIM2, FOXL2, GATA2, GATA3, IL-7R, IRF1, KIT, MAPK6, MAPKAPK3, RAB11B, STMN1, TNFRSF7 (CD27), CLK1 and PRKARI B in a biological sample from said animal following administration of the agent to the subject, or in a biological sample from an animal after contacting the sample with the agent,

(iii) providing a reference expression profile associated with the expression of one or more nucleic acids encoding a polypeptide selected from BIRC5, CALM1, CAMK2G, CaMKIINalpha, DC-UbP, FAIM2, FOXL2, GATA2, GATA3, IL-7R, IRF1, KIT, MAPK6, MAPKAPK3, RAB11B, STMN1, TNFRSF7 (CD27), CLK1 and PRKARI B in a biological sample from an animal exhibiting protective immunity; wherein increased similarity of the second expression profile to the reference expression profile, relative to the first expression profile to the reference expression profile, is indicative that the agent is capable of inducing protective immunity.

The screening methods mentioned herein may be employed either with a single test compound/agent or a plurality or library (e.g. a combinatorial library) of test compounds. In the latter case, synergistic effects provided by combinations of compounds may also be identified and characterized. The above-mentioned compounds may be used for inducing the level of memory T cells and/or promoting the survival of memory T cells, or may be used as lead compounds for the development and testing of additional compounds having improved specificity, efficacy and/or pharmacological (e.g. pharmacokinetic) properties. In an embodiment the compound may be a prodrug which is altered into its active form at the appropriate site of action, e.g. in lymphoid organs. In certain embodiments, one or a plurality of the steps of the screening/testing methods of the invention may be automated.

Expression levels may in general be detected by either detecting mRNA from the cells and/or detecting expression products, such as polypeptides and proteins. Expression of the transcripts and/or proteins encoded by the nucleic acids described herein may be measured by any of a variety of known methods in the art. In general, the nucleic acid sequence of a nucleic acid molecule (e.g., DNA or RNA) in a patient sample can be detected by any suitable method or technique of measuring or detecting gene sequence or expression. Such methods include, but are not limited to, polymerase chain reaction (PCR), reverse transcriptase-PCR (RT-PCR), in situ PCR, quantitative PCR (q-PCR), in situ hybridization, Southern blot, Northern blot, sequence analysis, microarray analysis, detection of a reporter gene, or other DNA/RNA hybridization platforms. For RNA expression, preferred methods include, but are not limited to: extraction of cellular mRNA and Northern blotting using labeled probes that hybridize to transcripts encoding all or part of one or more of the genes of this invention; amplification of mRNA expressed from one or more of the genes of this invention using gene-specific primers, polymerase chain reaction (PCR), quantitative PCR (q-PCR), and reverse transcriptase-polymerase chain reaction (RT-PCR), followed by quantitative detection of the product by any of a variety of means; extraction of total RNA from the cells, which is then labeled and used to probe cDNAs or oligonucleotides encoding all or part of the genes of this invention, arrayed on any of a variety of surfaces; in situ hybridization; and detection of a reporter gene. The term “quantifying” or “quantitating” when used in the context of quantifying transcription levels of a gene can refer to absolute or to relative quantification. Absolute quantification may be accomplished by inclusion of known concentration(s) of one or more target nucleic acids and referencing the hybridization intensity of unknowns with the known target nucleic acids (e.g., through generation of a standard curve). Alternatively, relative quantification can be accomplished by comparison of hybridization signals between two or more genes, or between two or more treatments to quantify the changes in hybridization intensity and, by implication, transcription level.

Methods to measure protein expression levels of selected genes of this invention, include, but are not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), flow cytometry, and assays based on a property of the protein including but not limited to DNA binding, ligand binding, or interaction with other protein partners.

Methods for normalizing the level of expression of a gene are well known in the art. For example, the expression level of a gene of the present invention can be normalized on the basis of the relative ratio of the mRNA level of this gene to the mRNA level of a housekeeping gene or the relative ratio of the protein level of the protein encoded by this gene to the protein level of the housekeeping protein, so that variations in the sample extraction efficiency among cells or tissues are reduced in the evaluation of the gene expression level. A “housekeeping gene” is a gene the expression of which is substantially the same from sample to sample or from tissue to tissue, or one that is relatively refractory to change in response to external stimuli. A housekeeping gene can be any RNA molecule other than that encoded by the gene of interest that will allow normalization of sample RNA or any other marker that can be used to normalize for the amount of total RNA added to each reaction. For example, the GAPDH gene, the G6PD gene, the actin gene, ribosomal RNA, 36B4 RNA, PGK1, RPLP0, or the like, may be used as a housekeeping gene.

Methods for normalizing/calibrating the level of expression of a gene are well known in the art. For example, the expression of a gene can be calibrated using reference samples, which are commercially available. Examples of reference samples include, but are not limited to: Stratagene® QPCR Human Reference Total RNA, Clontech™ Universal Reference Total RNA, and XpressRef™ Universal Reference Total RNA. Other methods are also described in Steinhoff and Vingron, Brief Bioinform. 2006 7(2):166-77; Fujita A. et al., BMC Bioinformatics. 2006. 7:469; and Tallat A M et al., Nucleic Acids Res. (2002). 30(20):e104, which are hereby incorporated by reference in their entireties.

Further, the normalization and calibration of gene expression may be performed in a straightforward manner for predictive models that involve pairs of predictive genes in competitive relationships, i.e. ratio of gene 1 over gene 2 in a predictive gene pair, obviating the need for additional reference genes. Instead of reporting the level of a predictive gene with respect to a separate housekeeping gene and/or reference sample, the level of predictive gene 1 with respect to predictive gene 2 directly provides for a relative expression measurement ratio with high information content.

Gene expression profiling or monitoring is a useful way to distinguish between cells that express different phenotypes. For example, cells that are derived from different organs/tissues, have different ages or different physiological states. In an embodiment, gene expression profiling can distinguish between different types or subsets of memory T cells. In an embodiment, gene expression profiling can distinguish between different types of immune responses, for example a protective versus a non-protective immune response.

Expression profile: One measurement of cellular constituents that is particularly useful in the present invention is the expression profile. As used herein, an “expression profile” comprises measurement of the relative abundance of one or more cellular constituents. Such measurements may include RNA or protein abundances or activity levels. An expression profile involves providing a pool of target nucleic acid molecules or polypeptides, hybridizing the pool to an array of probes immobilized on predetermined regions of a surface, and quantifying the hybridized nucleic acid molecules or proteins. The expression profile can be a measurement, for example, of the transcriptional state or the translational state of the cell. See U.S. Pat. Nos. 6,040,138, 6,013,449 and 5,800,992, which are hereby incorporated by reference in their entirety.

Similarity, with respect to gene expression profiles, means that the genes whose expression is measured exhibit the same trend in expression, and is not limited to absolute equivalence in expression levels. For example, two different samples in which a given gene shows a higher expression than an internal control would be considered to have “similar” expression profiles. In an embodiment, at least one gene exhibits the same trend in expression. In an embodiment, at least two genes exhibit the same trend in expression. In an embodiment, at least three genes exhibit the same trend in expression. In an embodiment, at least four genes exhibit the same trend in expression. In an embodiment, at least five genes exhibit the same trend in expression. In an embodiment, at least ten genes exhibit the same trend in expression. In an embodiment, at least twenty genes exhibit the same trend in expression. In an embodiment, at least fifty genes exhibit the same trend in expression.

Nucleic acid arrays are particularly useful for detecting the expression of the genes of the present invention. The production and application of high-density arrays in gene expression monitoring have been disclosed previously in, for example, PCT Publication No. WO 97/10365; PCT Publication No. WO 92/10588; U.S. Pat. No. 6,040,138; U.S. Pat. No. 5,445,934; or PCT Publication No. WO 95/35505, all of which are incorporated herein by reference in their entireties. Also for examples of arrays, see Hacia et al., Nature Genetics 14:441; Lockhart et al., Nat. Biotechnol. 14:1675-1680; and De Risi et al., Nature Genetics 14:457, each of which is incorporated by reference in its entirety. In general, in an array, an oligonucleotide, a cDNA, or genomic DNA, that is a portion of a known gene, occupies a known location on a substrate. A nucleic acid target sample is hybridized with an array of such oligonucleotides and then the amount of target nucleic acids hybridized to each probe in the array is quantified. One preferred quantifying method is to use confocal microscope and fluorescent labels. The Affymetrix GeneChip™ Array system (Affymetrix, Santa Clara, Calif.) and the Atlas™ Human cDNA Expression Array system are particularly suitable for quantifying the hybridization; however, it will be apparent to those of skill in the art that any similar systems or other effectively equivalent detection methods can also be used. In a particularly preferred embodiment, one can use the knowledge of the genes described herein to design novel arrays of polynucleotides, cDNAs or genomic DNAs for screening methods described herein. Such novel pluralities of polynucleotides are contemplated to be a part of the present invention and are described in detail below.

Suitable nucleic acid samples for screening on an array contain transcripts of interest or nucleic acids derived from the transcripts of interest (e.g., transcripts derived from the genes highly expressed in T_{CM} of the present invention). As used herein, a nucleic acid derived from a transcript refers to a nucleic acid for whose synthesis the mRNA transcript or a subsequence thereof has ultimately served as a template. Thus, a cDNA reverse transcribed from a transcript, an RNA transcribed from that cDNA, a DNA amplified from the cDNA, an RNA transcribed from the amplified DNA, etc., are all derived from the transcript and detection of such derived products is indicative of the presence and/or abundance of the original transcript in a sample. Thus, suitable samples include, but are not limited to, transcripts of the gene or genes, cDNA reverse transcribed from the transcript, cRNA transcribed from the cDNA, DNA amplified from the genes, RNA transcribed from amplified DNA, and the like. Preferably, such a sample is a total RNA preparation of a biological sample (e.g., peripheral blood mononuclear cells or PBMCs, immune cells, immune cell subpopulations such as memory T cells). More preferably in some embodiments, such a nucleic acid sample is the total mRNA isolated from such a biological sample.

Methods of isolating total mRNA are well known to those of skill in the art. In one embodiment, the total nucleic acid is isolated from a given sample using, for example, an acid guanidinium-phenol-chloroform extraction method and polyA and mRNA is isolated by oligo dT column chromatography or by using (dT)n magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ad. Greene Publishing and Wiley-Interscience, New York (1987)). Also, kits for the isolation of total RNA or mRNA are commercially available (e.g., Qiagen RNeasy Mini Kit, New England BioLabs polyA Spin™ mRNA isolation kit).

In general, typical biological samples include, but are not limited to, sputum, serum, lymphatic fluid, blood, blood cells (e.g., peripheral blood mononuclear cells), tissue or fine needle biopsy samples, urine, peritoneal fluid, colostrums, breast milk, fetal fluid, tears, and pleural fluid, or cells therefrom. In embodiments, the determination of expression levels is performed using peripheral blood mononuclear cells, such as immune cells, such as 4⁺ and CD8+ T cells. In embodiments, the determination of expression levels is performed using CD4 or CD8 T cell subsets, such as central memory or effector memory T cells.

In further embodiments, the invention relates to the use of nucleic acid(s) (e.g., a probe(s)) which is substantially homologous/identical or substantially complementary (e.g., for hybridization under moderately stringent or stringent conditions) to a nucleic acid sequence encoding one or more genes selected from the group consisting of HLA-G, MAL, NGFRAP1, HRMT1L2, ATXN3, TNFRSF7 (CD27), ING1, E2F4, RELA, TOSO, INDO, SFRP4, PABPC1, ARL7, PIM2, TAP1, CD37, LPPR4, IMPDH2, LOC112476, TGFBR2, CCNL1, GRK5, Stat5a, RALA, CSTB, SNF1LK, CAV1, MYO1E, B2M, NFIC, SYT6, RRM1, OAS1, IMPDH2, DMGDH, PNRC2, LIMS1, PARVG, FYN, LILRA2, FTL, SOCS1, PF4, ERG, IFIT1, NCOR2, IL16, TCIRG1, PITPNB, PABPC4, MAN2A1, SPN, TNFRSF8, RFX2, RGS13, LTA4H, S100A8, TCF3, TIAM1, CART, PPP2R2C, PIAS4, PRKCQ, NME2, SLC2A3, ATF4, IL2RG, COL3A1, PPM1D, SEC23A, LIMK2, BAT3, RGS10, STAT6, RASL12, CLQG, GPR18, NOTCH3, C1orf38, BTF3, CCL19, PES1, C1QA, ZNF593, TNF-a, POLD2, DTYMK, E2F1, STAU, IFNGR2, NRG1, TNFSF7, JARIDLA, BLR1, PLCL2, MKI67, IDUA, FEZ1, MAPKAPK5, DLC1, MAP4K2, VAV3, BATF, BIRC6, CEP2, DDAH2, PLK4, GTF2F2, FADS1, FHIT, SPOCK, TLK1, DDX5, NGFR, FYB, USP10, TCF7, RAMP1, AGPAT5, EDA, PPP3CC, HNRPK, TPR, CHUK, ANXA1, SMARCA4, CLK1, CCL3, CALM3, ALOX5, LCN2, NUP88, LKLF, AKT2, BIRC5, CALM1, CAMK2G, CaMKIINalpha, CLK1, GREG, DC-UbP, FAIM2, FOXL2, GATA2, GATA3, IL-7R, IRF1, KIT, KMO, CNTROB/LIP8, MAPK6, MAPKAPK3, PRKARI B, RAB11B, STMN1, STX1B2, STXBP5, GAS-7, XIAP, GADD45, DUSP1, PTEN, and SOCS2, a complement thereof, or a portion thereof.

“Homology” and “homologous” refers to sequence similarity between two peptides or two nucleic acid molecules. Homology can be determined by comparing each position in the aligned sequences. A degree of homology between nucleic acid or between amino acid sequences is a function of the number of identical or matching nucleotides or amino acids at positions shared by the sequences. As the term is used herein, a nucleic acid sequence is “homologous” to another sequence if the two sequences are substantially identical and the functional activity of the sequences is conserved (as used herein, the term ‘homologous’ does not infer evolutionary relatedness). Two nucleic acid sequences are considered “substantially identical” if, when optimally aligned (with gaps permitted), they share at least about 50% sequence similarity or identity, or if the sequences share defined functional motifs. In alternative embodiments, sequence similarity in optimally aligned substantially identical sequences may be at least 60%, 70%, 75%, 80%, 85%, 90% or 95%. As used herein, a given percentage of homology between sequences denotes the degree of sequence identity in optimally aligned sequences. An “unrelated” or “non-homologous” sequence shares less than 40% identity, though preferably less than about 25% identity, with any of the nucleic acids encoding the above-mentioned genes.

“Substantially complementary” nucleic acids are nucleic acids in which the complement of one molecule is substantially identical to the other molecule. Two nucleic acid or protein sequences are considered substantially identical if, when optimally aligned, they share at least about 70% sequence identity. In alternative embodiments, sequence identity may for example be at least 75%, at least 80%, at least 85%, at least 90%, or at least 95%. Optimal alignment of sequences for comparisons of identity may be conducted using a variety of algorithms, such as the local homology algorithm of Smith and Waterman, 1981, Adv. Appl. Math 2: 482, the homology alignment algorithm of Needleman and Wunsch, 1970, J. Mol. Biol. 48:443, the search for similarity method of Pearson and Lipman, 1988, Proc. Natl. Acad. Sci. USA 85: 2444, and the computerised implementations of these algorithms (such as GAP, BESTFIT, FASTA and TFASTA in the Wisconsin Genetics Software Package, Genetics Computer Group, Madison, Wis., U.S.A.). Sequence identity may also be determined using the BLAST algorithm, described in Altschul et al., 1990, J. Mol. Biol. 215:403-10 (using the published default settings). Software for performing BLAST analysis may be available through the National Center for Biotechnology Information (through the internet at www.ncbi.nlm.nih.gov). The BLAST algorithm involves first identifying high scoring sequence pairs (HSPs) by identifying short words of length W in the query sequence that either match or satisfy some positive-valued threshold score T when aligned with a word of the same length in a database sequence. T is referred to as the neighbourhood word score threshold. Initial neighbourhood word hits act as seeds for initiating searches to find longer HSPs. The word hits are extended in both directions along each sequence for as far as the cumulative alignment score can be increased. Extension of the word hits in each direction is halted when the following parameters are met: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program may use as defaults a word length (W) of 11, the BLOSUM62 scoring matrix (Henikoff and Henikoff, 1992, Proc. Natl. Acad. Sci. USA 89: 10915-10919) alignments (B) of 50, expectation (E) of 10 (or 1 or 0.1 or 0.01 or 0.001 or 0.0001), M=5, N=4, and a comparison of both strands. One measure of the statistical similarity between two sequences using the BLAST algorithm is the smallest sum probability (P(N)), which provides an indication of the probability by which a match between two nucleotide or amino acid sequences would occur by chance. In alternative embodiments of the invention, nucleotide or amino acid sequences are considered substantially identical if the smallest sum probability in a comparison of the test sequences is less than about 1, preferably less than about 0.1, more preferably less than about 0.01, and most preferably less than about 0.001.

An alternative indication that two nucleic acid sequences are substantially complementary is that the two sequences hybridize to each other under moderately stringent, or preferably stringent, conditions. Hybridisation to filter-bound sequences under moderately stringent conditions may, for example, be performed in 0.5 M NaHPO₄, 7% sodium dodecyl sulfate (SDS), 1 mM EDTA at 65° C., and washing in 0.2×SSC/0.1% SDS at 42° C. (see Ausubel, et al. (eds), 1989, Current Protocols in Molecular Biology, Vol. 1, Green Publishing Associates, Inc., and John Wiley & Sons, Inc., New York, at p. 2.10.3). Alternatively, hybridization to filter-bound sequences under stringent conditions may, for example, be performed in 0.5 M NaHPO₄, 7% SDS, 1 mM EDTA at 65° C., and washing in 0.1×SSC/0.1% SDS at 68° C. (see Ausubel, et al. (eds), 1989, supra). Hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y.). Generally, stringent conditions are selected to be about 5° C. lower than the thermal melting point for the specific sequence at a defined ionic strength and pH.

In an embodiment, it is desirable to amplify the nucleic acid sample prior to hybridization. One of skill in the art will appreciate that whatever amplification method is used, if a quantitative result is desired, a method is used that maintains or controls for the relative frequencies of the amplified nucleic acids to achieve quantitative amplification. Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. The high-density array may then include probes specific to the internal standard for quantification of the amplified nucleic acid. Other suitable amplification methods include, but are not limited to polymerase chain reaction (PCR) Innis, et al., PCR Protocols. A guide to Methods and Application. Academic Press, Inc. San Diego, (1990)), ligase chain reaction (LCR) (see Wu and Wallace, Genomics, 4: 560, Landegren, et al., Science, 241: 1077 and Barringer, et al., Gene, 89: 117), transcription amplification (Kwoh, et al., Proc. Natl. Acad. Sci. USA, 86: 1173), and self-sustained sequence replication (Guatelli, et al, Proc. Nat. Acad. Sci. USA, 87: 1874).

Another embodiment of the present invention relates to one or more polynucleotide probes for the detection of the expression of genes that are associated with memory T cell survival.

A “probe” is meant to include a nucleic acid oligomer that hybridizes specifically to a target sequence in a nucleic acid or its complement, under conditions that promote hybridization, thereby allowing detection of the target sequence or its amplified nucleic acid. Detection may either be direct (i.e, resulting from a probe hybridizing directly to the target or amplified sequence) or indirect (i.e., resulting from a probe hybridizing to an intermediate molecular structure that links the probe to the target or amplified sequence). A probe's “target” generally refers to a sequence within an amplified nucleic acid sequence (i.e., a subset of the amplified sequence) that hybridizes specifically to at least a portion of the probe sequence by standard hydrogen bonding or “base pairing.” Sequences that are “sufficiently complementary” allow stable hybridization of a probe sequence to a target sequence, even if the two sequences are not completely complementary. A probe may be labeled or unlabeled.

The polynucleotide probe(s) of the invention comprise, consist(s) of, or consist(s) essentially of, one or more polynucleotide probes that are complementary to RNA transcripts, or nucleotides derived therefrom, of at least one nucleic acid sequence that has been identified herein as being differentially expressed in T_{CM}, and is therefore distinguished from previously known nucleic acid arrays and primer sets. The plurality of polynucleotides within the above-limitation includes at least one or more polynucleotide probes (e.g., at least 1, 2, 3, 4, 5, 6, and so on, in whole integer increments, up to the maximum number of possible probes) that are complementary to RNA transcripts, or nucleotides derived therefrom, of at least one gene, and preferably, at least 2 or more genes identified by the present inventors. Such genes are selected from any of the genes listed in the tables provided herein and can include any number of genes, in whole integers (e.g., 1, 2, 3, 4, . . . ). Multiple probes can also be used to detect the same gene or to detect different splice variants of the same gene. In an aspect, each of the polynucleotides is at least 5 nucleotides in length. In an aspect, the polynucleotide probe(s) consist(s) of at least one polynucleotide probes, wherein each polynucleotide probe is at least 5 nucleotides in length, and wherein each polynucleotide probe is complementary to an RNA transcript, or nucleotide derived therefrom, of a gene selected from the group consisting HLA-G, MAL, NGFRAP1, HRMT1L2, ATXN3, TNFRSF7 (CD27), ING1, E2F4, RELA, TOSO, INDO, SFRP4, PABPC1, ARL7, PIM2, TAP1, CD37, LPPR4, IMPDH2, LOC112476, TGFBR2, CCNL1, GRK5, Stat5a, RALA, CSTB, SNF1LK, CAV1, MYO1E, B2M, NFIC, SYT6, RRM1, OAS1, IMPDH2, DMGDH, PNRC2, LIMS1, PARVG, FYN, LILRA2, FTL, SOCS1, PF4, ERG, IFIT1, NCOR2, IL16, TCIRG1, PITPNB, PABPC4, MAN2A1, SPN, TNFRSF8, RFX2, RGS13, LTA4H, S100A8, TCF3, TIAM1, CART, PPP2R2C, PIAS4, PRKCQ, NME2, SLC2A3, ATF4, IL2RG, COL3A1, PPM1D, SEC23A, LIMK2, BAT3, RGS10, STAT6, RASL12, CLQG, GPR18, NOTCH3, C1orf38, BTF3, CCL19, PES1, C1QA, ZNF593, TNF-a, POLD2, DTYMK, E2F1, STAU, IFNGR2, NRG1, TNFSF7, JARIDLA, BLR1, PLCL2, MKI67, IDUA, FEZ1, MAPKAPK5, DLC1, MAP4K2, VAV3, BATF, BIRC6, CEP2, DDAH2, PLK4, GTF2F2, FADS1, FHIT, SPOCK, TLK1, DDX5, NGFR, FYB, USP10, TCF7, RAMP1, AGPAT5, EDA, PPP3CC, HNRPK, TPR, CHUK, ANXA1, SMARCA4, CLK1, CCL3, CALM3, ALOX5, LCN2, NUP88, LKLF, AKT2, BIRC5, CALM1, CAMK2G, CaMKIINalpha, CLK1, GREG, DC-UbP, FAIM2, FOXL2, GATA2, GATA3, IL-7R, IRF1, KIT, KMO, CNTROB/LIP8, MAPK6, MAPKAPK3, PRKARI B, RAB11B, STMN1, STX1B2, STXBP5, GAS-7, XIAP, GADD45, DUSP1, PTEN, and SOCS2. In another aspect, the polynucleotide probe(s) comprise(s) polynucleotides that are complementary to an RNA transcript, or a nucleotide derived therefrom, of at least two genes mentioned above. In another aspect, the polynucleotide probe(s) comprises polynucleotide probes that are complementary to an RNA transcript, or a nucleotide derived therefrom, of at least five genes, at least 10 genes, at least 25 genes, at least 50 genes, or up to all of the genes mentioned above.

In accordance with the present invention, an isolated polynucleotide, or an isolated nucleic acid molecule, is a nucleic acid molecule that has been removed from its natural milieu (i.e., that has been subject to human manipulation), its natural milieu being the genome or chromosome in which the nucleic acid molecule is found in nature. As such, “isolated” does not necessarily reflect the extent to which the nucleic acid molecule has been purified, but indicates that the molecule does not include an entire genome or an entire chromosome in which the nucleic acid molecule is found in nature. The polynucleotides useful in the polynucleotide probes of the present invention are typically a portion of a gene (sense or non-sense strand) of the present invention that is suitable for use as a hybridization probe or PCR primer for the identification of a full-length gene (or portion thereof) in a given sample (e.g., a peripheral blood cell sample). An isolated nucleic acid molecule can include a gene or a portion of a gene (e.g., the regulatory region or promoter), for example, to produce a reporter construct according to the present invention. An isolated nucleic acid molecule that includes a gene is not a fragment of a chromosome that includes such gene, but rather includes the coding region and regulatory regions associated with the gene, but no additional genes naturally found on the same chromosome. An isolated nucleic acid molecule can also include a specified nucleic acid sequence flanked by (i.e., at the 5′ and/or the 3′ end of the sequence) additional nucleic acids that do not normally flank the specified nucleic acid sequence in nature (i.e., heterologous sequences). Isolated nucleic acid molecule can include DNA, RNA (e.g., mRNA), or derivatives of either DNA or RNA (e.g., cDNA). Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein. Preferably, an isolated nucleic acid molecule of the present invention is produced using recombinant DNA technology (e.g., polymerase chain reaction (PCR) amplification, cloning) or chemical synthesis.

The minimum size of a nucleic acid molecule or polynucleotide of the present invention is a size sufficient to form a probe or oligonucleotide primer that is capable of forming a stable hybrid with the complementary sequence of a nucleic acid molecule encoding the natural protein (e.g., under moderately stringent, or stringent conditions) (e.g. incubation at 65° C. in DIG Easy Hyb solution (Roche), 50 μg of yeast tRNA and 50 μg of calf thymus DNA) or to otherwise be used as a target in an assay or in any therapeutic method discussed herein. If the polynucleotide is an oligonucleotide probe or primer, the size of the polynucleotide can be dependent on nucleic acid composition and percent homology or identity between the nucleic acid molecule and a complementary sequence as well as upon hybridization conditions per se (e.g., temperature, salt concentration, and formamide concentration). The minimum size of a polynucleotide that is used as an oligonucleotide probe or primer is at least about 5 nucleotides in length, and preferably ranges from about 5 to about 50 or about 500 nucleotides or greater (1000, 2000, etc.), including any length in between, in whole number increments (i.e., 5, 6, 7, 8, 9, 10, . . . 33, 34, . . . 256, 257, . . . 500 . . . 1000 . . . ), and more preferably from about 10 to about 40 nucleotides, and most preferably from about 15 to about 40 nucleotides in length. There is no limit, other than a practical limit, on the maximal size of a nucleic acid molecule of the present invention, in that the nucleic acid molecule can include a portion of a protein-encoding sequence or a nucleic acid sequence encoding a full-length protein.

In an embodiment, the polynucleotide probes are conjugated to detectable markers. Detectable labels suitable for use in the present invention include any composition detectable by spectroscopic, photochemical, biochemical, immunochemical, electrical, optical or chemical means. Useful labels in the present invention include biotin for staining with labeled streptavidin or avidin conjugate, magnetic beads (e.g., Dynabeads™), fluorescent dyes (e.g., fluorescein, texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. Preferably, the polynucleotide probes are immobilized on a substrate.

In one embodiment, the polynucleotide probes are hybridizable array elements in a microarray or high density array. Nucleic acid arrays are well known in the art and are described for use in comparing expression levels of particular genes of interest, for example, in U.S. Pat. No. 6,177,248, which is incorporated herein by reference in its entirety. Nucleic acid arrays are suitable for quantifying small variations in expression levels of a gene in the presence of a large population of heterogeneous nucleic acids. Knowing the identity of the genes set forth by the present invention, nucleic acid arrays can be fabricated either by de novo synthesis on a substrate or by spotting or transporting nucleic acid sequences onto specific locations of substrate. Nucleic acids are purified and/or isolated from biological materials, such as a bacterial plasmid containing a cloned segment of sequence of interest. It is noted that all of the genes identified by the present invention have been previously sequenced, at least in part, such that oligonucleotides suitable for the identification of such nucleic acids can be produced. The database accession number for each of the genes identified by the present inventors is provided in the tables of the invention. Suitable nucleic acids are also produced by amplification of template, such as by polymerase chain reaction or in vitro transcription.

One of skill in the art will appreciate that an enormous number of array designs are suitable for the practice of this invention. An array will typically include a number of probes that specifically hybridize to the sequences of interest. In addition, in a preferred embodiment, the array will include one or more control probes. The high-density array chip includes “test probes”. Test probes could be oligonucleotides having a minimum or maximum length as described above for other oligonucleotides. In another embodiment, test probes are double or single strand DNA sequences. DNA sequences are isolated or cloned from natural sources or amplified from natural sources using natural nucleic acids as templates, or produced synthetically. These probes have sequences complementary to particular subsequences of the genes whose expression they are designed to detect. Thus, the test probes are capable of specifically hybridizing to the target nucleic acid they are to detect.

Another embodiment of the present invention relates to reagents which specifically binds with the polypeptide, such as chemical agents, or natural products, or antibodies, or antigen binding fragments thereof, for the detection of the expression of genes differentially expressed in T_{CM}. The reagent consists of chemical agents, or natural products, or antibodies, or antigen binding fragments thereof, that selectively bind to proteins encoded by genes that are regulated in biological samples from transplant donors, and that can be detected as protein products using antibodies. In addition, the reagent comprises chemical agents, or natural products, or antibodies, or antigen binding fragments thereof, that selectively bind to proteins or portions thereof (peptides) encoded by one or more genes selected from HLA-G, MAL, NGFRAP1, HRMT1L2, ATXN3, TNFRSF7 (CD27), ING1, E2F4, RELA, TOSO, INDO, SFRP4, PABPC1, ARL7, PIM2, TAP1, CD37, LPPR4, IMPDH2, LOC112476, TGFBR2, CCNL1, GRK5, Stat5a, RALA, CSTB, SNF1LK, CAV1, MYO1E, B2M, NFIC, SYT6, RRM1, OAS1, IMPDH2, DMGDH, PNRC2, LIMS1, PARVG, FYN, LILRA2, FTL, SOCS1, PF4, ERG, IFIT1, NCOR2, IL16, TCIRG1, PITPNB, PABPC4, MAN2A1, SPN, TNFRSF8, RFX2, RGS13, LTA4H, S100A8, TCF3, TIAM1, CART, PPP2R2C, PIAS4, PRKCQ, NME2, SLC2A3, ATF4, IL2RG, COL3A1, PPM1D, SEC23A, LIMK2, BAT3, RGS10, STAT6, RASL12, CLQG, GPR18, NOTCH3, C1orf38, BTF3, CCL19, PES1, C1QA, ZNF593, TNF-a, POLD2, DTYMK, E2F1, STAU, IFNGR2, NRG1, TNFSF7, JARIDLA, BLR1, PLCL2, MKI67, IDUA, FEZ1, MAPKAPK5, DLC1, MAP4K2, VAV3, BATF, BIRC6, CEP2, DDAH2, PLK4, GTF2F2, FADS1, FHIT, SPOCK, TLK1, DDX5, NGFR, FYB, USP10, TCF7, RAMP1, AGPAT5, EDA, PPP3CC, HNRPK, TPR, CHUK, ANXA1, SMARCA4, CLK1, CCL3, CALM3, ALOX5, LCN2, NUP88, LKLF, AKT2, BIRC5, CALM1, CAMK2G, CaMKIINalpha, CLK1, GREG, DC-UbP, FAIM2, FOXL2, GATA2, GATA3, IL-7R, IRF1, KIT, KMO, CNTROB/LIP8, MAPK6, MAPKAPK3, PRKARI B, RAB11B, STMN1, STX1B2, STXBP5, GAS-7, XIAP, GADD45, DUSP1, PTEN, and SOCS2. In an aspect, the reagent consists of one or more antibodies, antigen binding fragments thereof, or antigen binding peptides, each of which selectively binds to a protein encoded by one or more of the above-mentioned genes.

According to the present invention, the phrase “selectively binds to” refers to the ability of a chemical agent, a natural product, an antibody, antigen binding fragment or binding partner (antigen binding peptide) to preferentially bind to specified proteins. More specifically, the phrase “selectively binds” refers to the specific binding of one protein to another molecule (e.g., chemical agent, natural product, an antibody, fragment thereof, or binding partner to an antigen), wherein the level of binding, as measured by any standard assay (e.g., an immunoassay, fluorescence), is statistically significantly higher than the background control for the assay. For example, when performing an immunoassay, controls typically include a reaction well/tube that contain chemical agent, natural product, antibody or antigen binding fragment alone (i.e., in the absence of antigen), wherein an amount of reactivity (e.g., non-specific binding to the well) by the chemical agent, natural product, antibody or antigen binding fragment thereof in the absence of the antigen is considered to be background. Binding can be measured using a variety of methods standard in the art including enzyme immunoassays (e.g., fluorescence, ELISA, immunoblot assays, etc.).

Isolated antibodies of the present invention can include serum containing such antibodies, or antibodies that have been purified to varying degrees. Whole antibodies of the present invention can be polyclonal or monoclonal. Alternatively, functional equivalents of whole antibodies, such as antigen binding fragments in which one or more antibody domains are truncated or absent (e.g., Fv, Fab, Fab′, or F(ab)₂ fragments), as well as genetically-engineered antibodies or antigen binding fragments thereof, including single chain antibodies or antibodies that can bind to more than one epitope (e.g., bi-specific antibodies), or antibodies that can bind to one or more different antigens (e.g., bi- or multi-specific antibodies), may also be employed in the invention.

Generally, in the production of an antibody, a suitable experimental animal, such as, for example, but not limited to, a rabbit, a sheep, a hamster, a guinea pig, a mouse, a rat, or a chicken, is exposed to an antigen against which an antibody is desired. Typically, an animal is immunized with an effective amount of antigen that is injected into the animal. An effective amount of antigen refers to an amount needed to induce antibody production by the animal. The animal's immune system is then allowed to respond over a pre-determined period of time. The immunization process can be repeated until the immune system is found to be producing antibodies to the antigen. In order to obtain polyclonal antibodies specific for the antigen, serum is collected from the animal that contains the desired antibodies (or in the case of a chicken, antibody can be collected from the eggs). Such serum is useful as a reagent. Polyclonal antibodies can be further purified from the serum (or eggs) by, for example, treating the serum with ammonium sulfate.

For diagnostic applications, the reagent (i.e., the antibodies or antigen binding fragments thereof) is either in a free state or immobilized on a solid support, such as a tube, a bead, a microarray or any other conventional support used in the field. Immobilization is achieved using direct or indirect means. Direct means include passive adsorption (non-covalent binding) or covalent binding between the support and the reagent. By “indirect means” is meant that an anti-reagent compound that interacts with a reagent is first attached to the solid support. Indirect means may also employ a ligand-receptor system, for example, where a molecule such as a vitamin is grafted onto the reagent and the corresponding receptor immobilized on the solid phase. This is illustrated by the biotin-streptavidin system. Alternatively, a peptide tail is added chemically or by genetic engineering to the reagent and the grafted or fused product immobilized by passive adsorption or covalent linkage of the peptide tail.

Such diagnostic agents may be included in a kit which also comprises instructions for use. The reagent is labeled with a detection means which allows for the detection of the reagent when it is bound to its target. The detection means may be a fluorescent agent such as fluorescein isocyanate or fluorescein isothiocyanate, or an enzyme such as horseradish peroxidase or luciferase or alkaline phosphatase, or a radioactive element such as ¹²⁵I or ⁵¹Cr.

The invention also features kits for assessing the efficacy of a vaccine or a treatment at inducing/maintaining T_{CM} and/or a protective immune response in a subject. The kits can include reagents for evaluating the expression or activity of nucleic acids (e.g., mRNAs) or proteins that play a role in the induction and/or maintenance of T_{CM}. Kits for evaluating expression of nucleic acids can include, for example, probes or primers that specifically bind a nucleic acid of interest (e.g., a nucleic acid, the expression of which correlates with the presence or absence of T_{CM} in a sample). The kits for evaluating nucleic acid expression can provide substances useful as standard (e.g., a sample containing a known quantity of a nucleic acid to which test results can be compared, with which one can assess factors that may alter the readout of a diagnostic test, such as variations in an enzyme activity or binding conditions). Kits for assessing nucleic acid expression can further include other reagents useful in assessing levels of expression of a nucleic acid (e.g., buffers and other reagents for performing PCR reactions, or for detecting binding of a probe to a nucleic acid). In addition to, or as an alternative, kits can include reagents for detecting proteins (e.g., antibodies). The kits can provide instructions for performing the assay used to evaluate gene expression instructions for determining risk based on the results of the assay. For example, the instructions can indicate that levels of expression of a gene of interest (e.g., relative to a standard or a control), correlate with the presence or absence of T_{CM}. Kits can also provide instructions, containers, and other reagents for obtaining and processing samples for analysis.

The invention further provides methods for developing personalized treatment plans. Information gained by way of the methods described above can be used to develop a personalized treatment plan for a subject (for example, a vaccinated or an immunodeficient subject). The methods can be carried out by, for example, using any of the methods of gene analysis described above and, in consideration of the results obtained, designing a treatment plan or a clinical course of action for the subject. If the levels of gene expression indicate that the subject has low levels of T_{CM}, the subject is a candidate for vaccination and/or treatment with an effective amount of immuno-stimulating agent. Depending on the level of gene expression or the gene expression profile, the recipient may require a treatment regime that is more or less aggressive than a standard regime, or it may be determined that the recipient is best suited for a standard regime. When so treated, one can treat or prevent complications associated with poor immune response. Conversely, a different result (i.e., a different level of expression of certain genes) may indicate that the subject has high levels of T_{CM} and/or shows immune protection and is not likely to experience an undesirable clinical outcome (e.g. being at risk of infection). In that event, the patient may avoid vaccination and/or treatment with immuno-stimulating agents (or require a less aggressive regime) and their associated side effects.

In embodiments, the herein-mentioned animal is a mammal, such as a human. With regard to for example screening assays, accepted laboratory animal model systems may be used, for example rodent systems (e.g., mouse, rat, ferret), rabbit, non-human primates, as well as others known in the art.

The present invention is illustrated in further details by the following non-limiting examples.

### EXAMPLES

### Example 1

### Material and Methods

Reagents and Abs. Recombinant human IL-2 was obtained trough the AIDS reagent depository at the NIH. IL-7 and IL-4 were purchased from R&D systems. The kinase inhibitors AKT-IV, STO-069, U0126 and Wedelolactone were obtained from Calbiochem. Etoposide was purchased from Sigma-Aldrich. CH11, Anti-Fas agonist antibodies, were obtained from Immunotech. All antibodies for flow cytometry were purchased from BD PharMingen, except for anti-CD45RA-ECD from Beckman Coulter and anti-CCR7-FITC from R&D systems. Anti-pFOXO3a S253, anti-pFOXO3a T32, anti-pan FOXO3a, anti-Bim, anti-PIM-1, anti-pGab2 T452, anti-pIKKα/β S176/180 and anti-pAKT S473-alexa 488 were purchased from Cell Signaling Technology Inc., anti-FasL (5G51) from Alexis Biochemical, anti-P130 (clone KAB40) from Sigma, anti-Gadd45a from Chemicon, and anti-PIM-2 and anti-pFOXO3a S315 were a gift from BD-PharMingen. HRP-conjugated goat anti-mouse and goat anti-rabbit IgG antibodies were obtained from BioRad Laboratories. Anti-Rab-27a is a home-made antibody raised in rabbits against a GST-Rab27 fusion protein.

Isolation of CD4⁺ T cell sub-populations. PBMCs from healthy subjects were isolated by a Ficoll-HyPaque (Pharmacia) density gradient. All donors signed informed consent forms approved by the CHEST hospital review board and the CR-CHUM. We first enriched for CD4⁺ T cells using negative immunomagnetic bead selection (AutoMACS™, Myltenii). Cells were then labeled with anti-CD4-APC-cy7, anti-CD45RA-ECD, anti-CD27-FITC and anti-CCR7-PE-cy7 and sorted into Naïve, T_{CM} and T_{EM}. Sorting was performed using a BDAria™ flow cytometer (BD Biosciences). The purity of the T_{CM} and T_{EM} sub-populations ranged from 96 to 99%. All procedures were done at 4° C. to minimize any changes in cell phenotype or gene expression.

RNA isolation, amplification and microarray hybridization. Sample RNA was extracted using an RNA extraction kit (Qiagen), then amplified using the MessageAmp RNA kit (Ambion) following the manufacturer's instructions. The amplified RNA (aRNA) was then verified for quality and quantity using the Agilent Bioanalyser and measuring the OD. For reverse transcription, 10-20 μg of total RNA or 0.1-0.5 μg of mRNA was mixed on ice with the following (total volume 40 μL): 8.0 μL 5× First Strand reaction buffer (Superscript II, Invitrogen), 1.5 μL AncT primer (5′-T20VN, 100 pmol/μl), 3.0 μL dNTP-dCTP (6.67 mM each of dATP, dGTP, dTTP), 1.0 μL 2 mM dCTP, 1.0 μL 1 mM Cyanine 3 or Cyanine 5-dCTP (NEN), 4.0 μL 0.1 M DTT, 1.0-5.0 ng Control RNA (artificial Arabadopsis transcripts) and nuclease-free water (up to a total volume of 40 μL). The mixture was incubated in the dark at 65° C. for 5 minutes, then at 42° C. for 5 minutes. 2 μL of reverse transcriptase (SuperScript II, Invitrogen) was added and the incubation was continued at 42° C. for 2 hours. To stop the reaction, the mixture was briefly centrifuge and place on ice. 4 μL of 50 mM EDTA (pH 8.0) and 2 μL of 10 N NaOH were added, followed by an incubation at 65° C. for 20 minutes to hydrolyse the RNA. 4 μL of 5 M acetic acid was then added. The labeled cDNA was then purified using CyScribe™ GFX™ purification columns (Amersham) according to the manufacturer's protocol. Following purification, the volume was reduced to 5 μL by evaporation using a SpeedVac® system. For hybridization, a hybridization solution was prepared according to the following: for each 100 μL of DIG Easy Hyb solution (Roche), 5 μL of yeast tRNA (Invitrogen; 10 mg/ml) and 5 μL of calf thymus DNA was added. The hybridization solution was then incubated at 65° C. for 2 minutes and cool to room temperature. The labeled cDNA sample was then incubated in 80 μL of hybridization solution at 65° C. for 2 minutes and cool to room temperature. The hybridization mixture was then pipetted directly onto a coverslip and the slide (UltraGAPS™ slides, Corning Inc.) “array-side” was put down on top of the coverslip. The human 19k cDNA array (Microarray Centre, University Health Network, Toronto, Canada), a single-spotted array containing 19,008 characterised and unknown human ESTs, was used for the studies presented herein. The slides were then put in hybridization chambers and incubated for 12-16 hours at 37° C. For washing, the coverslip were quickly but gently dipping the array in 1×SSC, and the slides were placed into a staining rack and into a staining dish (Evergreen Scientific through Diamed cat# E/S258-4100-000) with fresh 1×SSC. The slides were washed for 3 sets of 15 minutes each at 50° C. in clean slide staining boxes containing pre-warmed (at 50° C.) 1×SSC/0.1% SDS solution with gentle occasional agitation. After the washes were completed, the slides were rinsed twice in room temperature 1×SSC (plunging 4-6 times) and then in 0.1×SSC. The slides were then spinned dry at 600 rpm for 5 minutes in a slide box lined with Whatman® paper (Whatman, UK) and scanned. Experimental design, sample description and preparations, hybridizations, data analysis and annotations meet MIAME compliance.

Microarray data preprocessing. Microarrays were scanned at 16 bits using the ScanArray Express Scanner T (Packard Bioscience) at 10-μm resolution at 635 (R) and 532 (G) nm wavelengths for cy-5 and cy-3 respectively to produce image (tiff) files that were quantified using Genepix Pro™ 6.0 image analysis software (Molecular Devices Corporation). Bad spots were flagged manually according to their morphologies. The results were saved as Quantarray™ files (QAF), where the intensity values ranged from 0 to 216-1 (65535) units. The tiff and QAF files were compressed and archived for permanent storage and further analysis. The microarrays were then screened for quality, first by visual inspection of the array with flagging of poor quality spots, and second with automated scripts that scanned the quantified output files and measured overall density distribution on each channel and number of flagged spots. Box-plots, MA-plots, and density distribution plots were drawn and inspected. Each quantified output file was run though the following pre-processing steps using the R language and environment (http://www.r-project.org, Wit et al., 2004. Statistics for Microarrays: Design, Analysis and Inference. John Wiley and Sons Ltd, England. 1-265 pp.—Dalgaard, 2002. Introductory Statistics, R. Springer. 1-288 pp.; Maindonald et al., 2003. Data Analysis and Graphics, R. Cambridge University Press, Cambridge. 1-362 pp.; Everitt et al., 2006. A Handbook of Statistical Analyses, R. Chapman & Hall/CRC, Boca Raton, Fla. 1-304 pp.) and the Limma package (Smyth, Bioinformatics and Computational Biology Solutions using R and Bioconductor, 397-420). For minimum intensity filtering, R and G values were treated with a surrogate replacement policy for estimating sub-threshold values. For normalization within arrays, the raw merged R and G channels were lowess-normalized (grouped by print-tip) and transformed to log 2 ratios (Smyth, supra; and Fukunaga, Introduction to statistical pattern recognition, 1-592). The commensurability of average brightness between the arrays of a pool of arrays was then assured using zero-centering of log-distributions normalization. When both duplicate spots of a clone (gene) passed quality control, the average profile of the replicate clones was calculated and used as the representative profile for that gene. If only one of the clone duplicate spots passed quality control, only that profile was used in the downstream analysis. All data were then represented as log 10 (Red/Green) expression ratios for further analysis.

Selection of top 100 T_{CM}/T_{EM} discriminating genes. From the set of 19k microarray genes that passed QC criteria during preprocessing of microarray data, we retained only the genes for further analysis with T_{CM}/T_{EM} discriminating F-test p-values<0.01. From this set, a final subset of 100 genes was further manually selected according to known functions and pathways including apoptosis, cell cycle and signaling.

Principal Components Analysis (PCA). A data matrix comprising 13 T_{CM} and 13 T_{EM} samples (rows), and 100 “top” T_{CM}/T_{EM} discriminating genes (see above) was constructed. Using singular value decomposition of the data matrix, a standard PCA of the data's 100×100 covariance matrix was computed, each sample comprising 100 genes. PCA computed and plot generated by Ref GeneLinker Platinum™ V4.6 (Improved Outcomes Software Inc, Ontario, Canada).

Two-way hierarchical clustering. Hierarchical clustering was carried out over the same set of 26 samples and 100 genes as used for PCA. We used Pearson correlation as the similarity measure between genes and samples for clustering. Analyses were performed using GeneLinker Platinum™ V4.6 software.

Quantitative real-time PCR analysis. Changes in gene expression observed by array analyses were verified by low-density array performed on an Applied Biosystems detection system (Foster City, Calif.). Briefly, cDNA was synthesized from total RNA (1 μg per sample, n=4) in a reverse transcriptase (RT) reaction in 20 μl of 1× first-strand synthesis buffer (Invitrogen). Amplification of cDNA ( 1/20) was performed using SYBR Green PCR buffer (Perkin-Elmer, Wellesley, MA), containing 0.1 μM of specific primers. Before the samples were analyzed, standard curves of purified, target-specific amplicons were created. The mRNA expression for each gene was determined by comparing it with its respective standard curve. This measurement was controlled for RNA quality, quantity, and RT efficiency by normalizing it to the expression level of the GAPDH gene. Statistical significance was determined by use of normalized fold changes and ANOVA. The p-values were calculated using a two-tailed T-test, and assuming that the true variances were unknown.

Induction and quantification of apoptosis. Sorted cells were cultured in complete RPMI and then treated as indicated in the figure legends. Apoptotic cells were detected using annexin-V labeling according to the manufacturer's protocol (Biosource). The fluorescence signals were measured by flow cytometry using a BD LSRI flow cytometer (BD-Biosciences). Approximately 50,000-gated events were collected for each sample.

Western Blotting analysis. T_{CM} and T_{EM} were sorted as described above. Cells were washed twice with PBS and re-suspended in lysis buffer containing 50 mM NaF and 1 mM sodium pyrophosphate. Proteins from total cell extracts (10 μg) were separated on SDS-PAGE and electrotransfered onto PVDF membranes (Roche, Indianapolis, Ind.). Membranes were incubated overnight at 4° C. with specific antibodies as described in the figure legends. Detection of the immune complexes was performed using horseradish peroxidase (HRP)-conjugated goat anti-mouse (1:2500) or goat anti-rabbit IgG antibodies (1:3000). HRP activity was detected using an enhanced chemiluminescence detection procedure (ECL-plus detection system, Amersham Biosciences). Membranes were subsequently stripped and restained with an anti-actin Abs (1:10000). The expression level of actin was used to control for equal loading. Protein expression levels were expressed as a percentage of the highest signals obtained.

Intracellular staining. The cells were labeled with anti-CD4-Amcyan, anti-CD27-PB, anti-CD45RA-APCcy7 for 20 min at 4 degrees. The cells were fixed in 2% PFA for 15 min at RT and then incubated with anti-GrB-Alexa700, anti-Perforin-FITC or Anti-FASL-PE for 20 min at RT in 0.5% saponin (in PBS). Analysis was performed on gated-T_{CM} and -T_{EM}. Around 20,000-gated events were collected on a BD LSRII flow cytometer (BD-Biosciences).

Proliferation assay. Sorted T_{CM} or T_{EM} were co-cultured with mature autologous dendritic cells (mDC) (ratio T/DC:40/1) in the presence of SEA (50 ng/ml) as previously described (Younes et al., 2003. J. Exp. Med. 198(12):1909-22.). After 1 to 15 days of co-culture, cells were labeled with anti-CD4 and anti-TCRVβ22. For the analysis, cells were gated on 4⁺ T cells and approximately 150,000-gated events were collected on a LSRII cytometer.

Flow cytometry analysis of STAT5a and AKT phosphorylation status. PBMCs were resuspended at 20 millions/ml in RPMI and incubated for 30 min in the presence of CCR7-FITC abs (20 μl/million cells) at room temperature. The cells were then washed and re-suspended at a cell concentration of 5 millions per ml in PBS and stimulated for 15 min at 37° C. in the presence of IL-2 (100 U/ml) or IL-7 (10 ng/ml). Following stimulation, the cells were fixed for 10 min at 37 degrees using cytofix buffer (BD Biosciences), pelleted and then permeabilized in PERM-III buffer (BD Biosciences) for 30 min on ice. The cells were then washed twice in Staining buffer (BD Biosciences) and rehydrated for 30 min on ice in the staining buffer. Cells were then labeled with anti-CD4-APCcy7, anti-CD45RA-ECD, anti-CD27-PE and anti-pSTAT5a (Y694)-Alexa647 specific antibodies for 30 min at room temperature. For the analysis, the cells were gated on T_{CM} and T_{EM}. An average of 20,000-gated events was collected on LSRII cytometer. For CD28 cross-linking, the cells were re-suspended at 10 million/ml in the presence of CD28 (2 μg/ml) for 30 min on ice. The cells were then washed twice in PBS and subsequently stimulated by cross-linking with rabbit anti-mouse Igs (20 μg/ml) (Biosource) in 25 μl pre-warmed medium for 15 min. The cells were then fixed and permeabilized as described above and labeled with CD4-APCcy7, CD45RA-ECD, CD27-PE, pAKT S473-Alexa 488. Flow cytometry analysis was performed on gated T_{CM} and T_{EM}. Around 20,000-gated events were collected on a BD LSRII cytometer.

### Example 2

### Functional and Phenotypic Characterization of CD4⁺ T_{CM} and T_{EM}

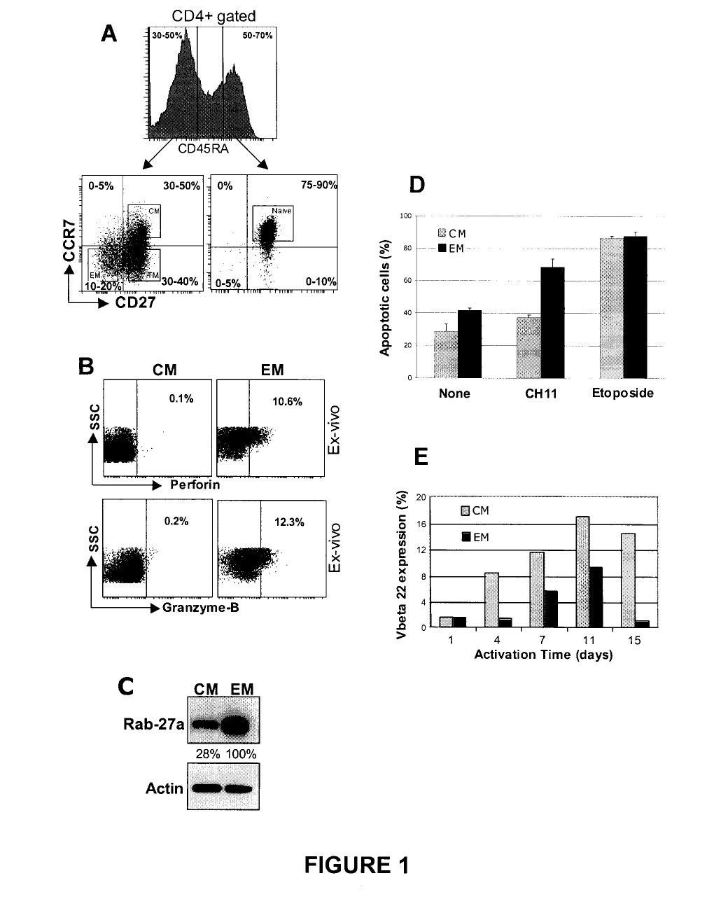

Memory T cell subsets were sorted by flow cytometry from whole PBMC isolated from 13 healthy donors based on CD45RA, CD27 and CCR7 expression. Naïve cells are characterized as CD45RA+, CD27+ and CCR7+; T_{CM} cells (Central Memory) have the following phenotype: CD45RA−, CD27+ and CCR7+, and T_{TM} cells (“Transitory” Memory) are CD45RA−, CD27+ and CCR7−; while T_{EM} cells (Effector Memory) are defined by the lack of expression of these three markers (CD45RA−, CD27− and CCR7−) (Seder, R. A., and R. Ahmed. 2003. Nat Immunol 4:835-842) (FIG. 1A). All T_{CM} (>95%) expressed CD28, CD62L and CD95 (Fas). T_{EM} were also homogeneously CD28+ and CD95+, albeit only 30-40% expressed CD62L (data not shown). The ex-vivo sorted T_{EM} subpopulation expressed the effector cytotoxic molecules Granzyme B and perforin, while these two molecules were undetectable in T_{CM} (FIG. 1B). T_{EM} also showed higher (threefold) expression levels of Rab27a, a molecule involved in degranulation and cytotoxic effector function (15), than T_{CM} (FIG. 1C). Taken together, these results show that T_{EM} are functionally and phenotypically more differentiated than T_{CM}.

### Example 3

### T_{CM} CD4⁺ T Cells are resistant to Fas-Induced Apoptosis and Show enhanced proliferation capacity following Stimulation with Mature Dendritic Cells

We next determined the sensitivity to apoptosis of T_{CM} and T_{EM}. T_{CM} and T_{EM} sorted cells were cultured in the presence or absence of anti-Fas antibodies or etoposide for 24 hours (n=3). Annexin-V labeling showed a significant difference (p<0.007) in the capacity of T_{CM} to resist Fas-mediated apoptosis as compared to T_{EM} (FIG. 1D). Of note, T_{CM} cells are less prone to undergo spontaneous apoptosis (i.e., without any apoptotic inducers) (p<0.02) than the T_{EM} subset (FIG. 1D). Moreover, in response to etoposide, used as a non-specific apoptotic inducer, both T_{CM} and T_{EM} present similar sensitivity to apoptosis, thereby confirming that the apoptotic machinery is intact in both cell types. We also determined the capacity of purified T_{CM} and T_{EM} to proliferate and persist in a 15-day culture assay after stimulation with SEA-pulsed mature dendritic cells. Proliferation was determined by quantifying the expansion of the SEA-responsive TCRVβ22+ T cells. T_{CM} present a better expansion potential and can persist longer than T_{EM}, as demonstrated by a tenfold increase in the absolute number of SEA-responsive TCRVβ22+T cells in a 15-day culture period (FIG. 1E). Similar data were also generated using CFSE whereby T_{CM} undergo several more rounds of proliferation when compared to T_{EM} (data not shown). Collectively, these results demonstrate that CD4⁺ T_{CM} and T_{EM} subsets exhibit different capacities to proliferate, persist and undergo both spontaneous and Fas-induced apoptosis. These observations led us to investigate the cell survival pathways responsible for that resistance to cell death in T_{CM} and to characterize the differences in these pathways between T_{CM} and T_{EM}.

### Example 4

### Gene Expression Profiling Analysis of T_{CM} and T_{EM} Showed Differences in the Expression of Genes Associated with Survival Pathways

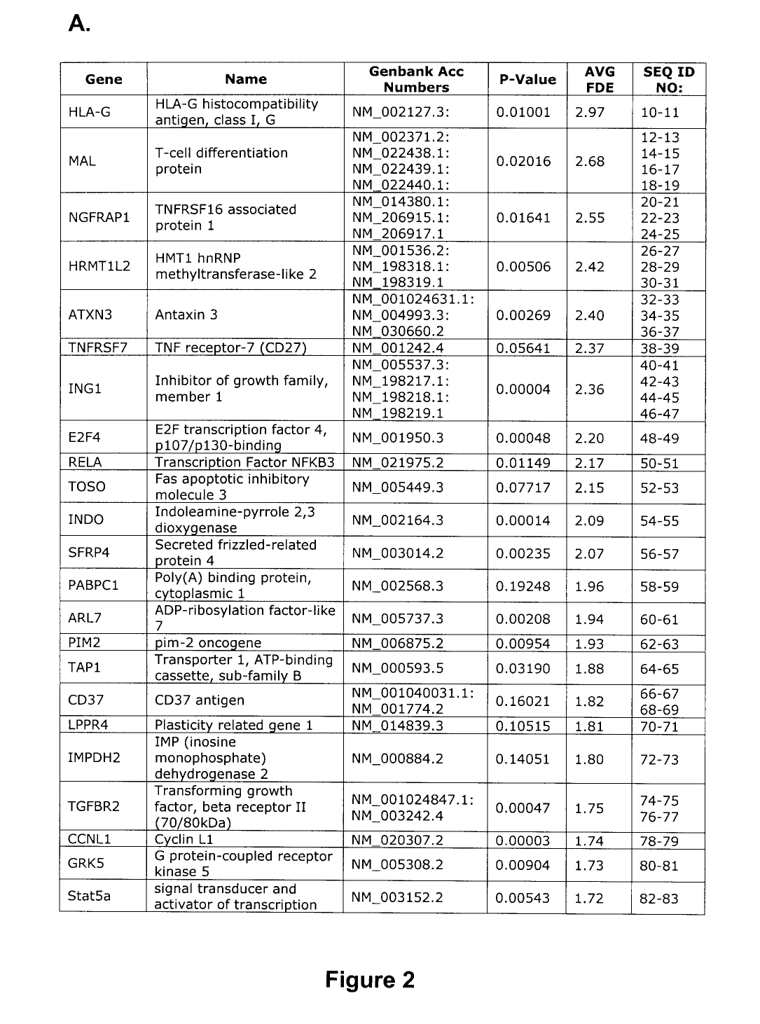

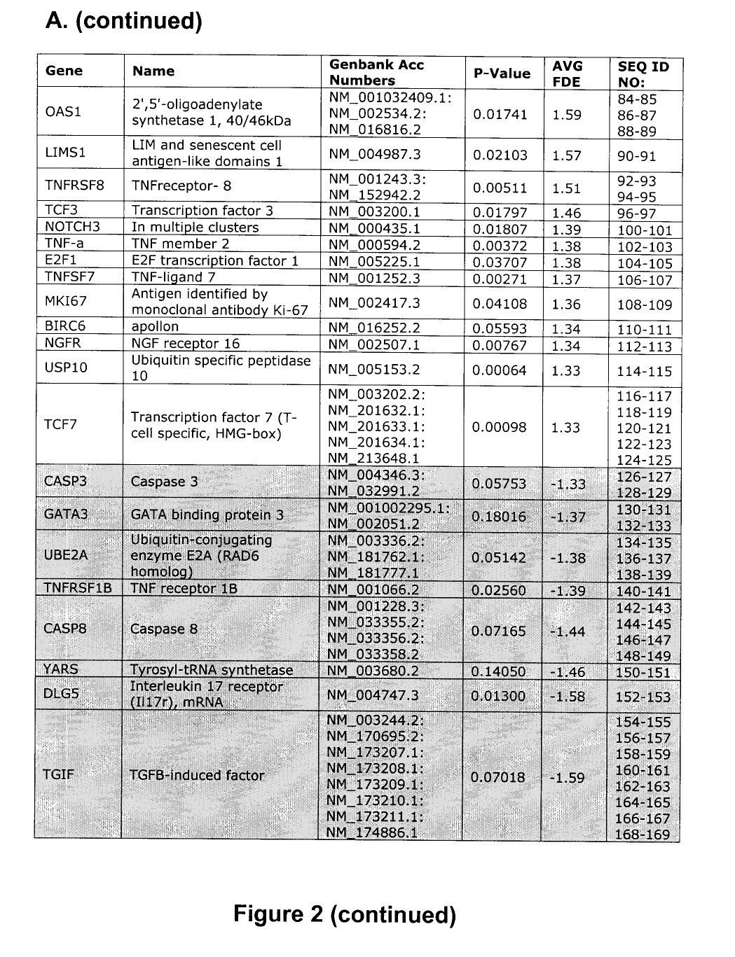

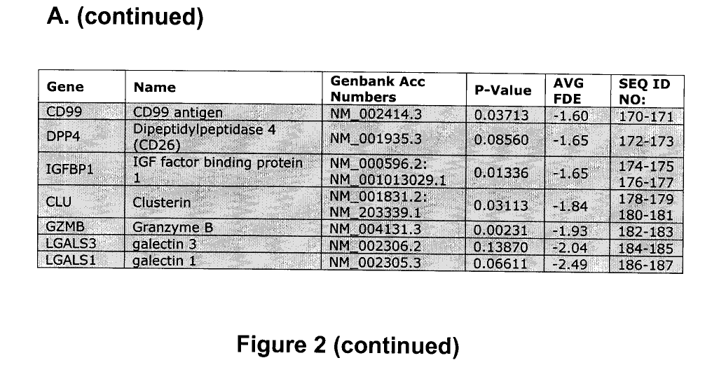

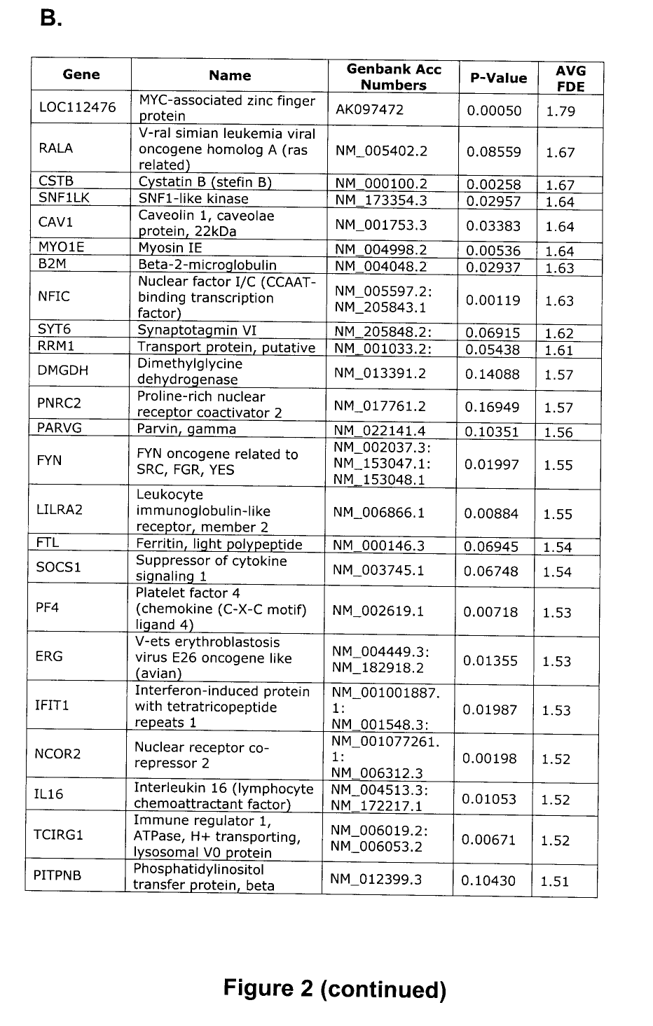

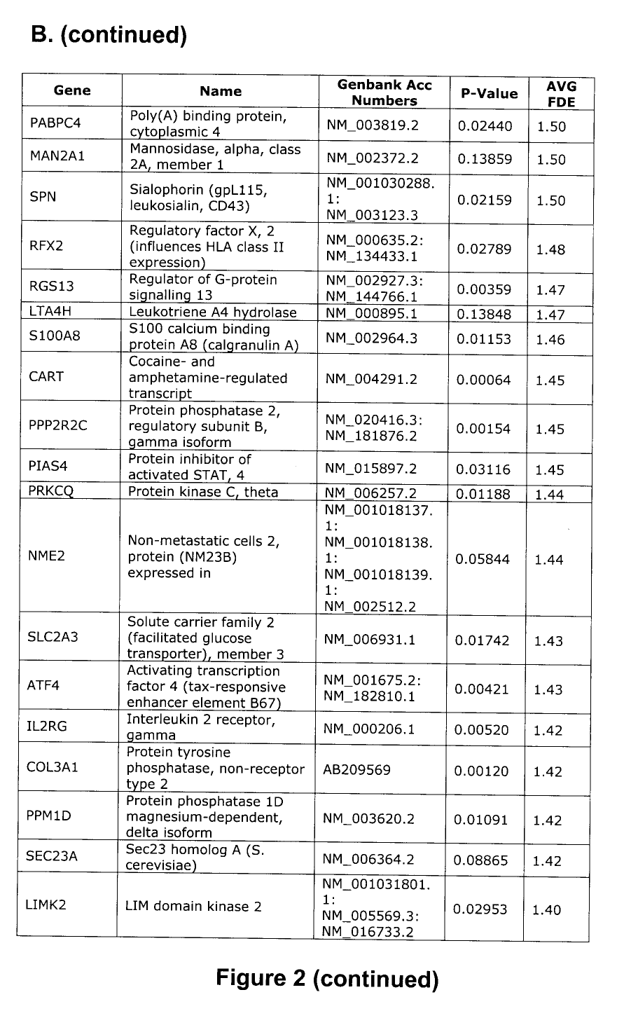

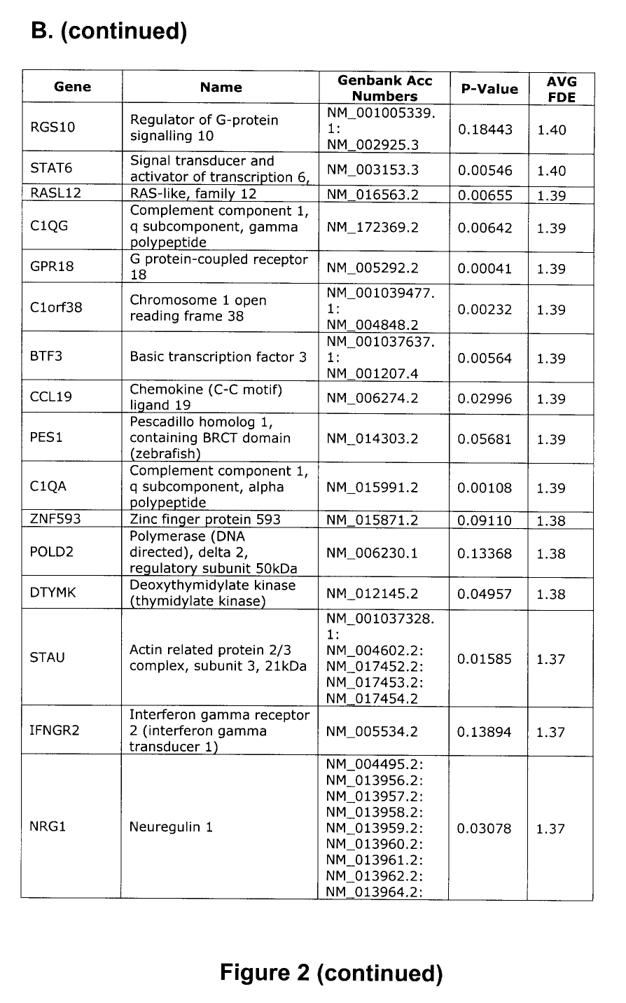

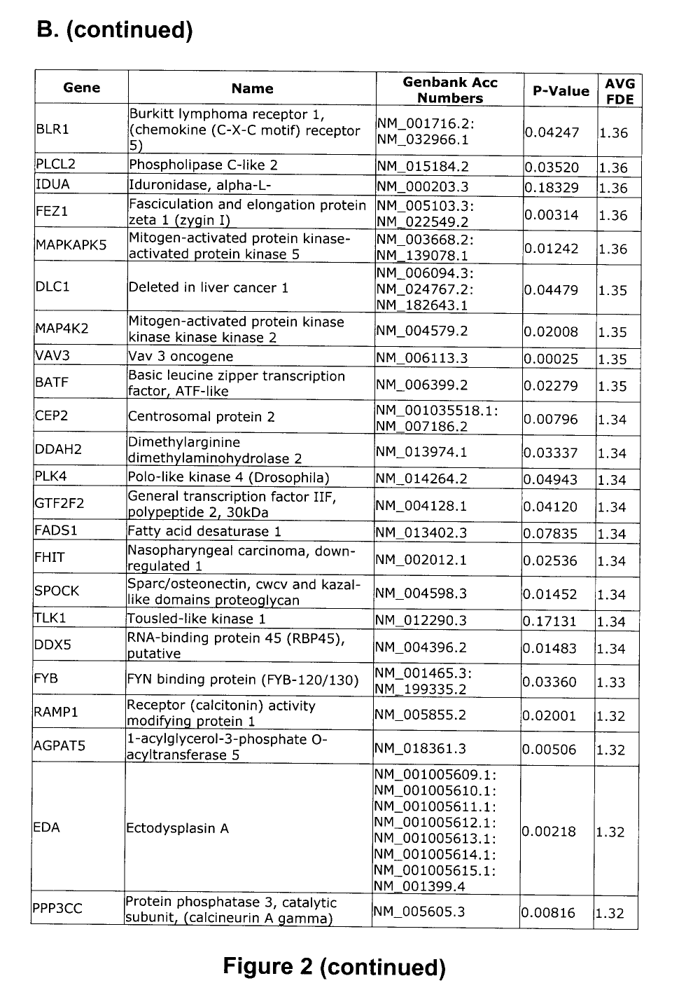

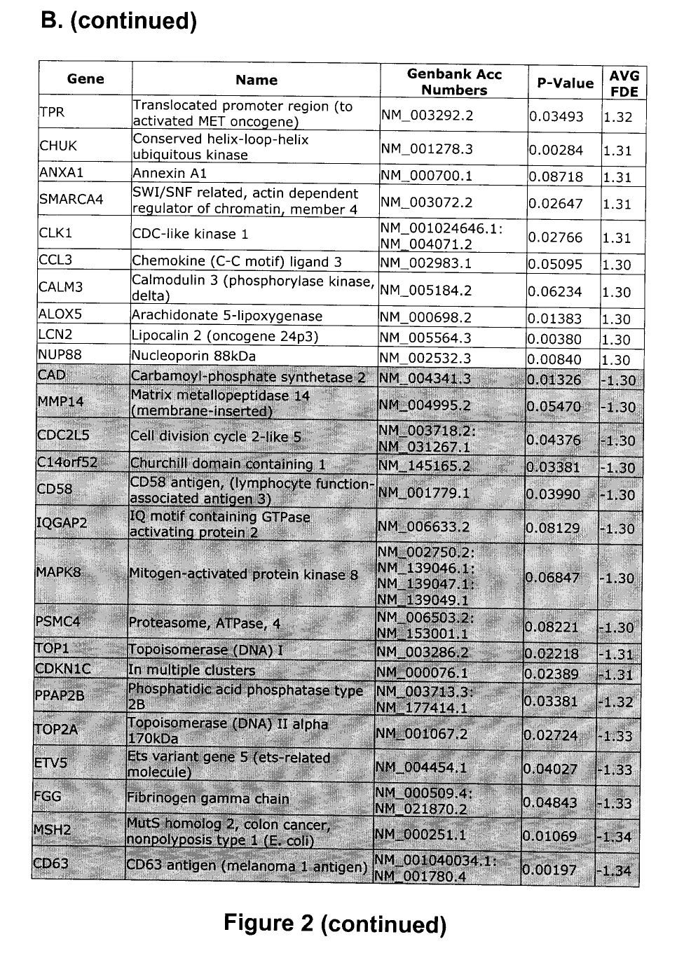

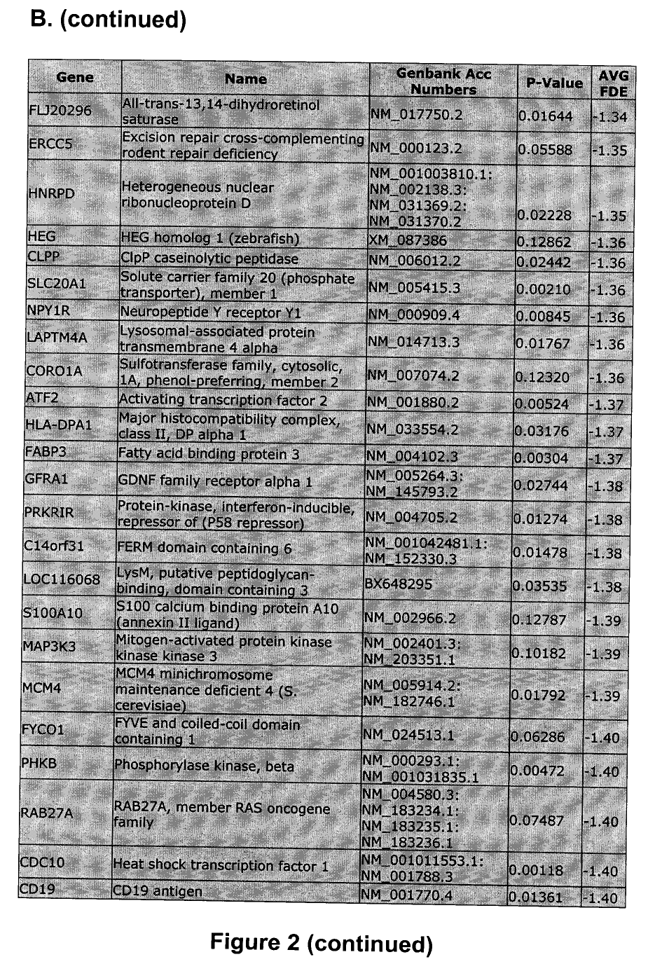

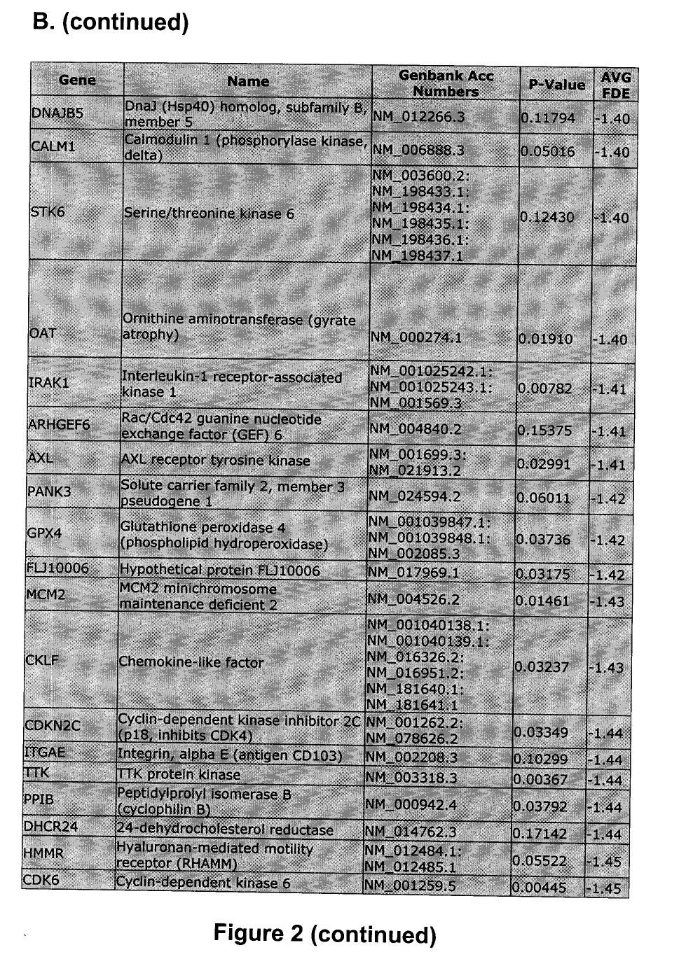

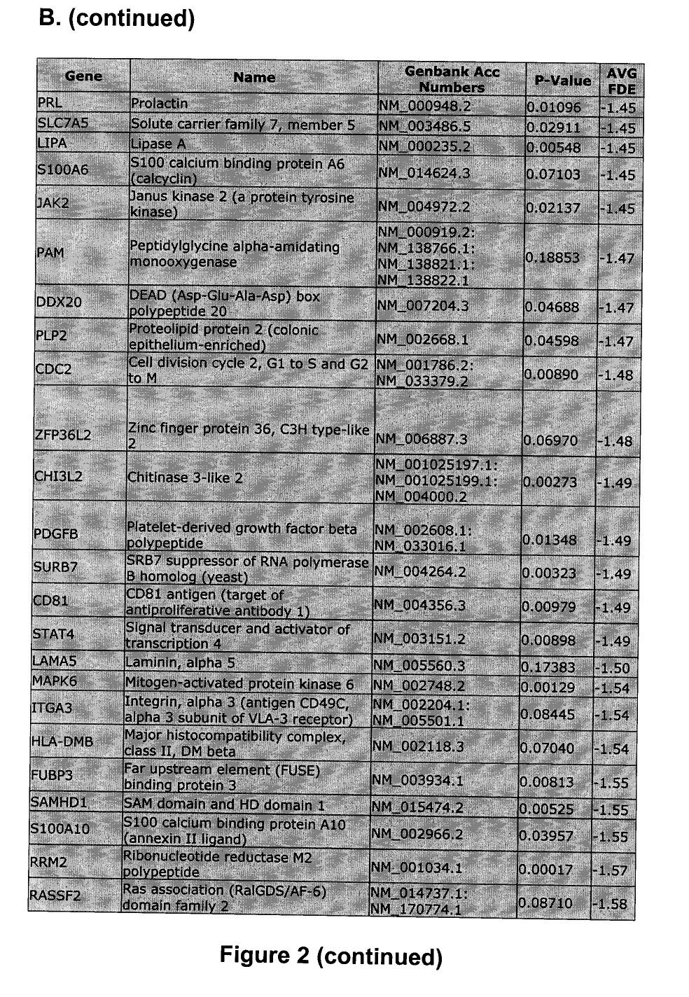

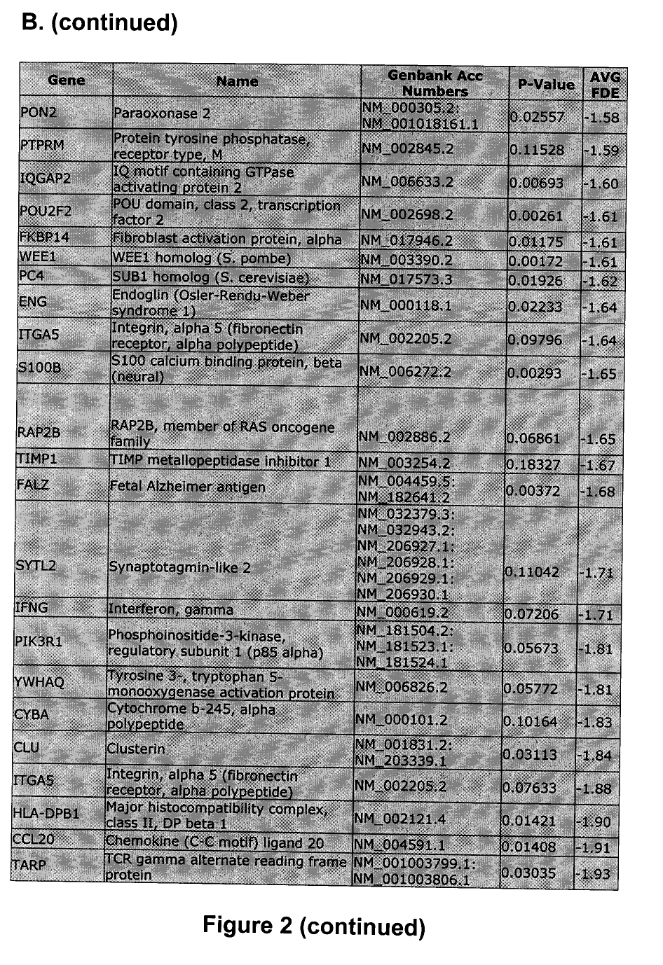

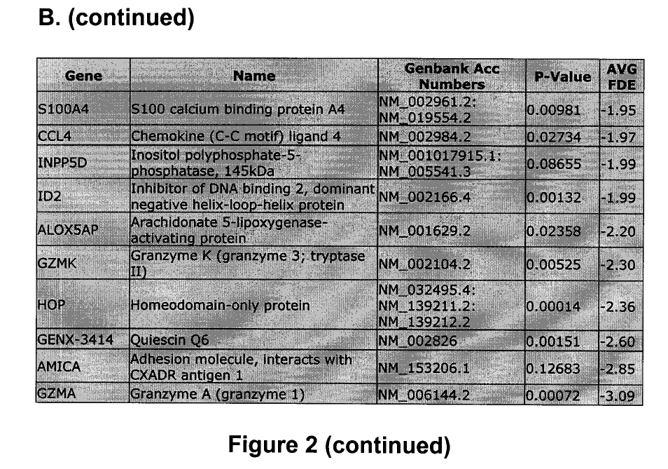

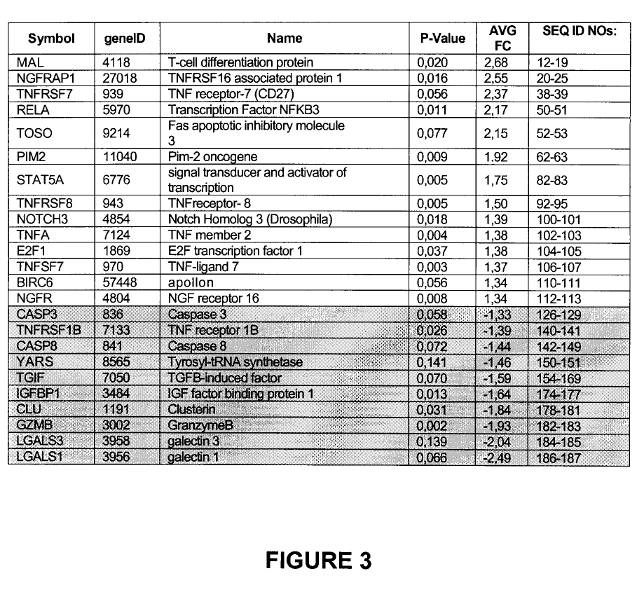

Using single gene searches, we then identified the genes that distinguished T_{CM} from T_{EM}. Genes selected using ANOVA, where p<0.05 or fold difference in expression >1.3 were considered significant. We identified more than 270 significant genes that distinguished both subsets (see FIGS. 2a and 2b). Within the selected genes, 6% were related to apoptosis, 9% to cell cycle/cell proliferation, and 7% to signaling. These genes also encompassed biological functions including homing/adhesion, gene expression regulation, immune response and transport. Apoptosis-related genes displaying a different expression profile when comparing T_{CM} and T_{EM} are listed in FIG. 3. T_{CM} expressed higher levels of TOSO, CD27, STAT5a, PIM-2, RelA, and Birc6 (Bruce) mRNA, all belonging to distinct anti-apoptotic pathways (Hitoshi, Y., et al., 1998. Immunity 8:461-471; Yan, B. et al., 2003. J Biol Chem 278:45358-45367; Gravestein, L. A. et al., 1998. Eur J Immunol 28:2208-2216; Grossmann, M. et al., 2000. Embo J 19:6351-6360; Hao, Y. et al., 2004. Nat Cell Biol 6:849-860), than their T_{EM} counterparts. In contrast, T_{EM} showed higher levels of expression of genes involved in the induction of apoptosis, including Caspase-8 and Caspase-3, as well as several proteins endowed with a pro-apoptotic function, such as Galactin-1 (LGALS1), Galactin-3 (LGALS3) (Hahn, H. P. et al., 2004. Cell Death Differ 11:1277-1286), Clusterin (Shannan, B. et al., 2006. Cell Death Differ 13:12-19), YARS (Wakasugi, K., and P. Schimmel. 1999. Science 284:147-151) and TGIF, a TGFp-targeted gene (Feng, X. H., and R. Derynck. 2005. Annu Rev Cell Dev Biol. 21:659-93). This expression profile suggested that T_{EM} contain an active pro-apoptotic machinery. On the other hand, several genes that promote cell survival were selectively expressed at high levels in T_{CM}, rendering them more resistant to apoptosis. Of note, we observed similar differences in the expression of several of the above-cited genes when comparing T_{CM} and T_{EM} in the CD8+T cell compartment (data not shown).

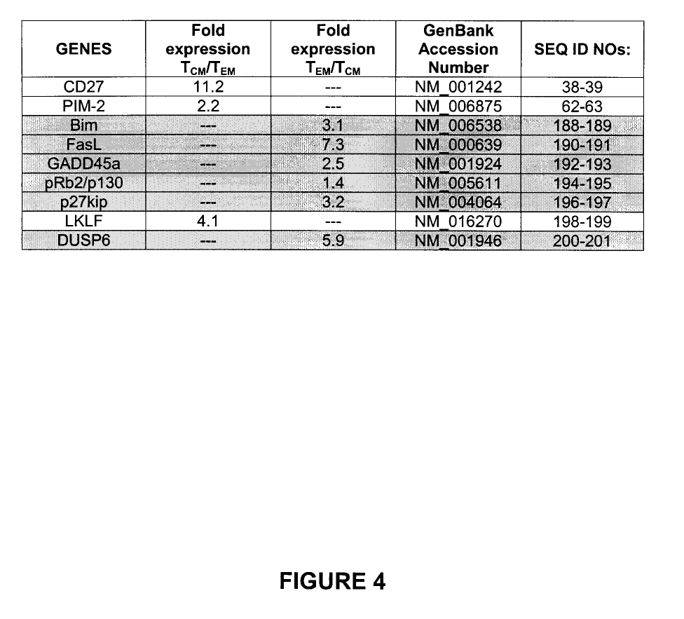

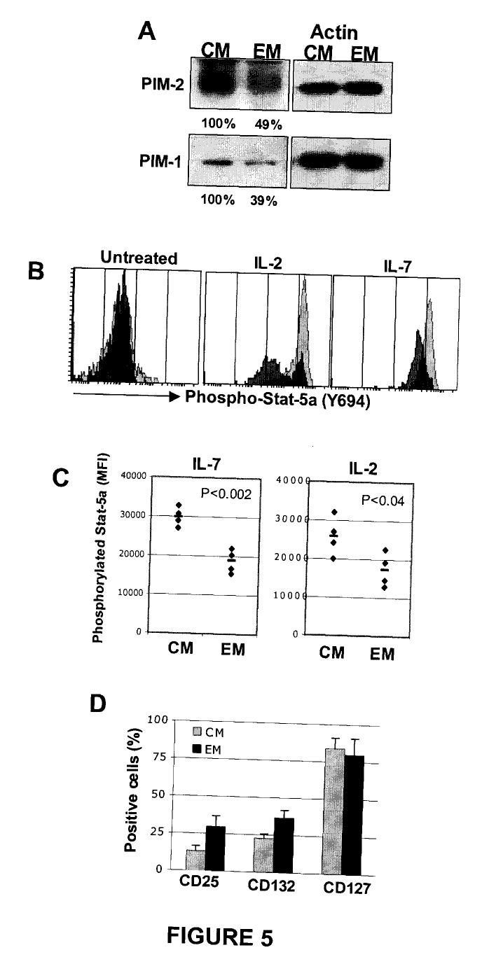

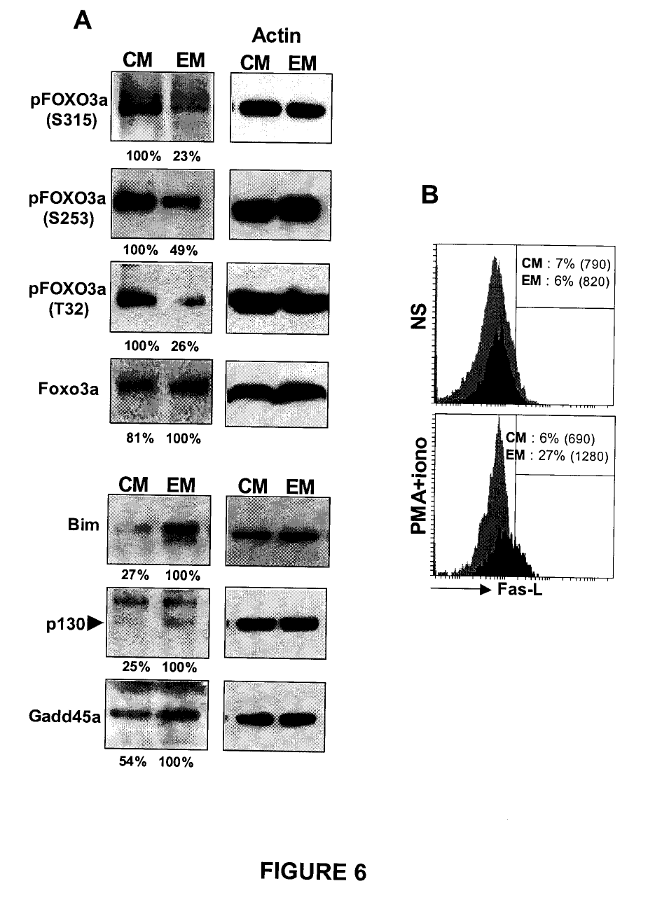

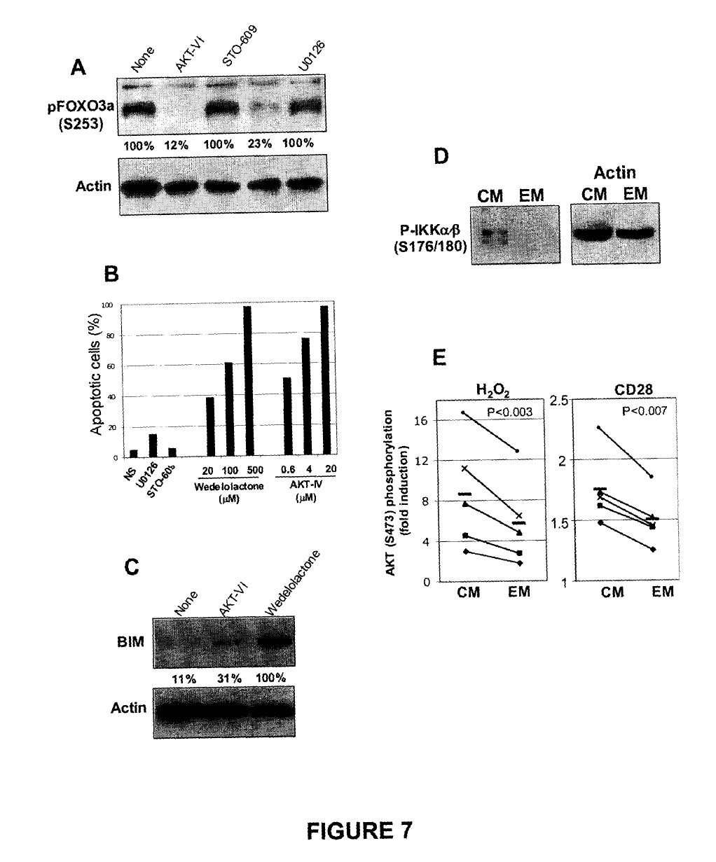

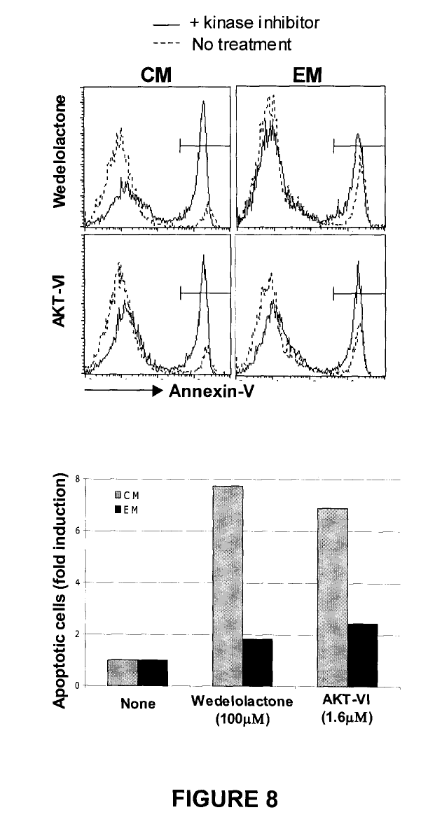

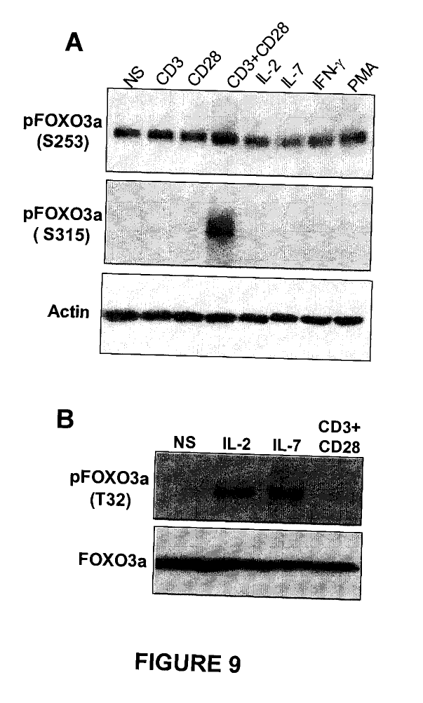

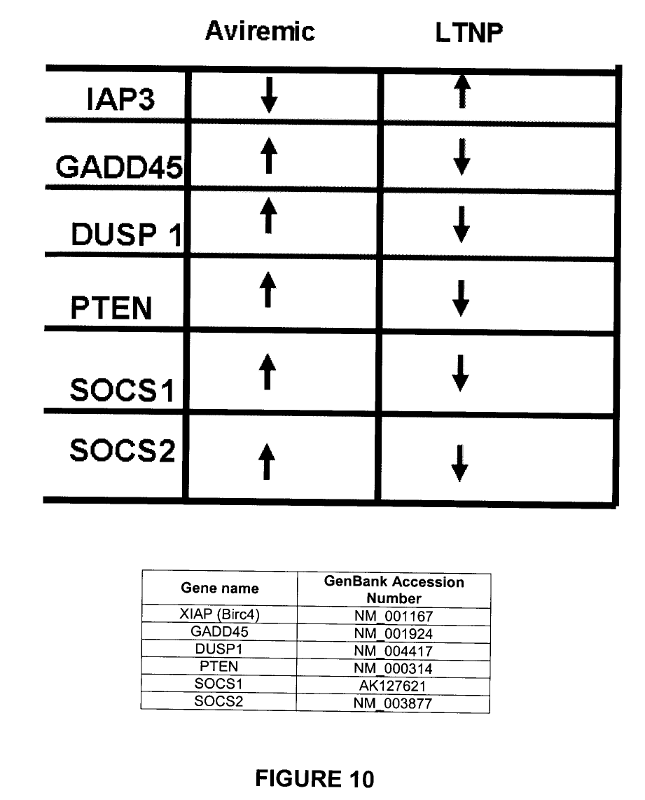

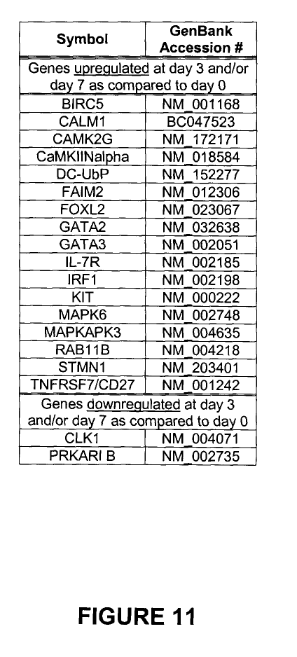

In the next set of experiments, we validated and detailed the gene array data by performing real-time RT-PCR on the same donor samples (FIG. 4). The results showed a significant increase in the FOXO3a transcriptional target pro-apoptotic genes, including Bim (Essafi, A. et al., 2005. Oncogene 24:2317-2329), FasL (Suhara, T. et al., 2002. Mol Cell Biol 22:680-691), and genes involved in cell cycle regulation including GADD45a (Tran, H. et al., 2002. Science 296:530-534) as well as pRb2/p13O, a member of the Retinoblastoma family (Kops, G. J. et al., 2002. Mol Cell Biol 22:2025-2036) in the T_{EM} subset (n=5). Furthermore, the RT-PCR data confirmed the upregulation of CD27 and the anti-apoptotic PIM-2 kinase (FIG. 4), as well as of TOSO and TGIF (data not shown). These results validated our gene array data and further suggested the involvement of STAT5a and FOXO3a signaling pathways in mediating the survival of T_{CM}.

### Example 5

### STAT5A Signaling Pathway is Functionally Upregulated in T_{CM}

STAT5a is a downstream effector of yc cytokines (Nosaka, T. et al., 1999. Embo J 18:4754-4765). We observed differential expressions of PIM-1 and PIM-2, two transcriptional targets of STAT5a in the ex-vivo T_{CM} subset. Indeed, T_{CM} showed twofold higher expression of both PIM-1 and PIM-2 than T_{EM} (n=3) (FIG. 5A). Because of the importance of the STAT5a pathway in the regulation of T-cell survival (Nosaka, T. et al., supra), we evaluated the ability of IL-2 and IL-7 to trigger the STAT5a signaling pathway in CD4⁺ T cell memory subsets. The phosphorylated form of STAT5a (Y694) (pSTAT5a) was quantified by flow cytometry. Basal pSTAT5a levels were similar in T_{CM} and T_{EM} (FIG. 5B). Both T_{CM} and T_{EM} upregulated pSTAT5a in response to a brief IL-7 treatment (FIG. 5B). However, the proportion of cells that up-regulated pSTAT5a was significantly higher (30%±6.5, p<0.002) in T_{CM} as compared to T_{EM} (FIG. 5C). Treatment with IL-2 also induced differential pSTAT5a levels (p<0.04) between T_{CM} and T_{EM}. Indeed, 90-100% of T_{CM} showed a phosphorylated STAT5a form, compared to 50-60% observed in T_{EM}. Of note, T_{EM} present a bimodal distribution of pSTAT5a in response to IL-2, indicating that T_{EM} are heterogeneous in terms of response to IL-2.

The differences in pSTAT5a levels were not due to differences in the levels of expression of IL-2 or IL-7 receptors. Indeed, the proportion of cells expressing CD127 (IL-7Rx), CD25 (IL-2Ra) and CD132 (yc chain) on T_{CM} were comparable to those on T_{EM} (FIG. 5D). Of note, CD122 (IL-2RP) was undetectable on ex-vivo T_{CM} and T_{EM} (data not shown). Although IL-2R is expressed on about 20% of T_{CM} as assess by cytometry, 100% of these cells are able to phosphorylate STAT5 in response to IL-2. This suggests that T_{CM} express undetectable levels of IL-2R that are sufficient to induce STAT5 signaling in response to IL-2. Taken together, these results indicate that the STAT5a pathway, as shown by the levels of pSTAT5 and its downstream effectors (PIM-1 and PIM-2), is differentially regulated between T_{CM} and T_{EM}. The observed differences suggest that T_{CM} display an enhanced capacity to mobilize the STAT5a pathway for their survival as compared to T_{EM}.

### Example 6

### Regulation of the FOXO3a Pathway in Memory 4⁺ T Cell Subsets

FOXO3a (Genbank accession Number NM_{—}001455, Anderson, M. J. et al., 1998. Genomics 47(2), 187-199) belongs to the forkhead family of transcription factors which are characterized by a distinct forkhead domain. FOXO3a transcriptional activity is regulated through direct phosphorylation. Once phosphorylated, FOXO3a is excluded from the nucleus and thus becomes transcriptionally inactive. FOXO3a controls the expression of several genes including FasL, Bim, Gadd45a, p27kip and p130 (Coffer, P. J., and B. M. Burgering. 2004. Nat Rev Immunol 4:889-899, Van Der Heide, L. P. et al., 2004. Biochem J 380:297-309). Our gene expression profiling analysis and RT-PCR data suggested the specific involvement of the FOXO3a pathway in T_{CM} survival. Therefore, we analyzed the phosphorylation status of FOXO3a in T_{CM} and T_{EM}. We observed that the levels of phosphorylated forms of FOXO3a (pFOXO3a) (phosphorylated at position S315 and/or S253 and/or T32) were reproducibly (n=5) more than twofold higher in ex-vivo T_{CM} as compared to T_{EM}. It is worth noting here that expression levels of total FOXO3a remained similar in the two memory subsets (FIG. 6A, upper panel). We then determined whether the observed decrease in FOXO3a phosphorylation levels observed in T_{EM} was associated with increased levels of FOXO3a transcriptional target proteins. Our results show that T_{EM} expressed threefold higher levels of Bim and p130 proteins and a 1.7-fold higher expression of GADD45a when compared to the T_{CM} compartment (FIG. 6A, bottom panel). FasL, whose mRNA transcript was clearly expressed at higher levels in T_{EM} than in T_{CM} (see FIG. 4), was undetectable in ex-vivo T_{CM} and T_{EM} when assayed by Western blot and flow cytometry (data not shown). However, upon T cell activation induced by PMA and ionomycin, FasL was selectively upregulated in T_{EM} (in around 30% of the T_{EM} subset), while it remained undetectable in T_{CM} (FIG. 6B). Taken together, our data show that a high expression of pFOXO3a observed in T_{CM} is associated with the low expression of pro-apoptotic proteins Bim, Gadd45a and p130, thereby favoring their resistance to apoptosis and consequently their long-term survival.

### Example 7

### Blocking of AKT and IKK Kinases Activity Prevents FOXO3A Phosphorylation Leading to T_{CM} Cell Death

To identify the kinases involved in the phosphorylation of FOXO3a in T_{CM}, we analyzed FOXO3a phosphorylation, in total CD4⁺ T cells (total CD4⁺ T cells were used due to the limiting amounts of T_{CM} and T_{EM} obtained after cell sorting) following treatment with specific kinase inhibitors. We used the pharmacological kinase inhibitors AKT-IV and Wedelolactone, which respectively inhibit AKT and IKK activities. We also tested two other kinase inhibitors: STO-609, specific for CamKK described as an upstream mediator of AKT (Soderling, T. R. 1999. Trends Biochem Sci 24:232-236); and the Mek1/2 inhibitor, U0126, used as an irrelevant kinase inhibitor. The results (FIG. 7A) clearly showed that treatment with the AKT and IKK inhibitors led to a specific and significant reduction in the levels of pFOXO3a (S253). The expression levels of pFOXO3a (S253) in CD4⁺ T cells was eightfold lower in the presence of the AKT-inhibitor and 4.5-fold lower in the presence of the IKK-inhibitor, as compared to untreated cells (FIG. 7A).

To confirm the importance of the phosphorylation of FOXO3a in memory T-cell survival, purified CD4⁺ T cells were treated with different kinase-inhibitors, and apoptosis was assessed by flow cytometry using Annexin-V labeling. FIG. 7B shows that the proportion of Annexin-V+ cells are increased in a dose-dependant fashion after exposing CD4⁺ T cells to AKT or IKK inhibitors. Moreover, we observed a significant upregulation of the levels of the pro-apoptotic molecule Bim, known to be a FOXO3a target, in cells treated with AKT or IKK inhibitors (three and eightfold respectively) (FIG. 7C). Of note, the AKT and IKK inhibitors did not change the levels of FasL expression when assayed by Western blot or flow cytometry (data not shown). These results indicated that the dephosphorylation of FOXO3a in CD4⁺ T cells was associated with Bim upregulation and apoptosis. None of the other kinase-inhibitors tested induced apoptosis in CD4⁺ T cells (FIG. 7B) even when used at much higher concentrations (data not shown). Taken together, these results show that among the kinase-inhibitors tested, only those able of inducing FOXO3a dephosphorylation have the capacity to induce CD4⁺ T cell apoptosis. It is thus likely that activated AKT and IKK promote CD4⁺ T cell survival, at least in part, by phosphorylating FOXO3a, thereby repressing its transcriptional activity and leading to the downregulation of the transcription of the pro-apoptotic molecule Bim.

To assess the importance of AKT and IKK in the survival of memory T cells, we evaluated the expression of the phosphorylated forms of these proteins in T_{CM} and T_{EM} subsets. First, pIKKα/β expression was assayed in ex-vivo sorted T_{CM} and T_{EM}. While pIKKα/β was expressed in T_{CM}, it was undetectable in T_{EM} (FIG. 7D). Second, since the phosphorylated form of AKT (pAKT) was undetectable by Western blot in ex-vivo sorted cells, we performed a PhosFlow analysis on PBMCs treated with H₂O₂ (known to induce phosphorylation of AKT). The results showed that the induction of pAKT (S473) was higher in T_{CM} than T_{EM} in response to H₂O₂ (FIG. 7E). More importantly, in response to CD28 triggering, T_{CM} cells presented a modest (about 20%), though consistent and significant increase (p<0.007) in AKT phosphorylation as compared to the T_{EM} (FIG. 7E). These results suggest that in resting T_{CM}, the constitutive activation of IKKα/β could maintain the level of FOXO3a phosphorylation. Moreover, CD28 triggering leads to higher levels of pAKT in T_{CM} and could also promote FOXO3a phosphorylation. Taken together, these results suggest that the phosphorylation levels of FOXO3a in T_{CM} can be maintained, both in their resting state and upon CD28 triggering, through the activation of IKK and AKT, respectively, thereby promoting T_{CM} survival.

The corollary of the above results is that the lack of FOXO3a phosphorylation could render T_{CM} susceptible to signals inducing cell death. To determine the implication of the AKT- and IKK-signaling pathways in the survival of T_{CM}, we sorted T_{CM} and T_{EM} and exposed them to AKT- or IKK inhibitors at their IC₅₀ (AKT-IV: 1.6 μM and wedelolactone: 100 μM). After 24 h of treatment, the proportion of apoptotic cells was quantified using Annexin-V labeling (FIG. 8, top panel). The treatment of T_{CM} with the IKK inhibitor resulted in an eightfold increase in Annexin-V+ cells, while only a twofold increase was observed in T_{EM}, all relative to untreated cells (n=2) (FIG. 8, bottom panel). Similar results were also observed when these subsets were exposed to the AKT inhibitor (FIG. 8). Experiments aimed at blocking FasL triggering, using Fas-Fc chimera, in response to AKT and IKK inhibitor-induced apoptosis did not prevent T_{CM} cell death (data not shown). These results indicate that abrogating the AKT and/or IKK pathways leads to apoptosis to a greater extent in T_{CM} as compared to T_{EM} confirming that these pathways are involved in the survival of T_{CM}. Moreover, the ability of AKT and IKK inhibitors to up-regulate Bim levels of expression (see FIG. 7C) without affecting FasL expression suggest that cell death induced by these kinase inhibitors could be achieved in part thought FOXO3a dephosphorylation and the subsequent increased expression of pro-apoptotic molecules such as Bim.

### Example 8

### TCR and IL-7 Triggering Phosphorylate Distinct Sites on FOXO3A

To identify the signals that trigger FOXO3a phosphorylation in CD4⁺ T cells, we quantified the levels of pFOXO3a (S253, S315 and T32) in response to CD3 and/or CD28 cross-linking, as well as IL-2, IL-7, IFN-γ and PMA treatment. pFOXO3a (S253) was easily detectable in ex-vivo CD4⁺ T cells, and none of the tested stimuli induced significant changes in the expression level of this phosphorylated form of FOXO3a (FIG. 9A). In contrast, FOXO3a phosphorylation on S315 was significantly induced in response to CD3+CD28 triggering. It is worth noting that CD3 or CD28 triggering alone did not lead to detectable FOXO3a phosphorylation (S315), suggesting that both signals synergize to induce FOXO3a phosphorylation at S315 (FIG. 9A, top panel, lanes 2, 3 and 4). None of the other tested inducers (including γc cytokines) led to FOXO3a (S315) phosphorylation. Interestingly, the levels of pFOXO3a at T32 were significantly increased (threefold) when CD4⁺ T cells were treated with IL-7. No induction of pFOXO3a (T32) was observed when cells were triggered with anti-CD3/CD28 or IL-2 (FIG. 9B). Taken together, these results indicate that TCR and IL-7 triggering induce specific FOXO3a phosphorylation at distinct sites (S315 and T32 respectively), suggesting that FOXO3a phosphorylation involves multiple signals.

### Example 9

### Comparison of the Expression of Selected Genes in T_{CM} Isolated from Aviremic HAART-Treated Hiv-Infected Individuals vs. Long-Term Non-Progressors (LTNPs)

PBMC obtained from LTNP and aviremic HAART-treated patients were sorted into T_{CM} and T_{EM} using CD27, CCR7 and CD45RA surface markers. Sorted cells were subjected to RNA isolation, amplification and gene array analysis. Long-term non-progressors (LTNPs) are subjects infected by HIV for more than 10 years and who naturally (i.e. in the absence of antiretroviral therapy) control HIV infection. Their CD4 T cell count remains relatively stable and they exhibit low HIV viral load (Pantaleo G. and Fauci A. S., 1995. Annu Rev Immunol, 13:487-512). Aviremic HAART-treated patients are HIV-infected patients who do not naturally control HIV infection in the absence of antiviral therapy, but who maintained low HIV viral loads when treated with HAART. FIG. 10 shows differences in the gene expression profile of T_{CM} from LTNPs versus aviremic HAART-treated HIV patients. T_{CM} from LTNP subjects show higher expression of IAP3 but lower expression of GADD45a, DUSP1, PTEN, SOCS1 and SOCS2 as compared to T_{CM} from aviremic HAART-treated subjects.

### Example 10

### Comparison of the Expression of Genes in Blood Samples Isolated Prior to and after Yellow Fever-Vaccination

Whole blood from Yellow Fever (YF)-vaccinated subjects was collected 14 days post-immunization. Whole blood from HIV infected individuals was collected during primary HIV infection (the first 6 months after the first positive diagnosis). Whole blood cells were lysed and their RNA was reverse transcribed to cDNA and subjected to gene array analysis using the method described in Example 1. The Yellow Fever 17D vaccine is a live-attenuated vaccine which induces efficacious and long-term protection against Yellow Fever infection in vaccinated individuals (Barrett A. D., 2001. Ann. N.Y. Acad. Sci., 951: 262-71) and therefore constitutes a good model for the induction of a protective immune response in humans. FIG. 11 depicts the genes whose expression in blood cells is significantly modulated after YF vaccination (3 and/or 7 days after vaccination).

Although the present invention has been described hereinabove by way of specific embodiments thereof, it can be modified, without departing from the spirit and nature of the subject invention as defined in the appended claims.

1. A method of identifying an agent capable of (a) inducing the level of memory T cells, (b) promoting the survival of memory T cells, or (c) both (a) and (b), comprising determining Foxo3a phosphorylation in the presence versus the absence of a test agent, wherein a higher level of phosphorylated Foxo3a in the presence of the agent is indicative that the agent is capable of (a) inducing the level of memory T cells, (b) promoting the survival of memory T cells, or (c) both (a) and (b).

2. The method according to claim 1, wherein said phosphorylation is at a Foxo3a residue corresponding to Thr32, Ser253, Ser315, or any combination thereof.

3. The method according to claim 1, wherein said memory T cell is a central memory T cell (T_{CM}).

4. A method of identifying an agent capable of (a) inducing the level of memory T cells, (b) promoting the survival of memory T cells, or (c) both (a) and (b), comprising determining the expression of one or more nucleic acids or polypeptides comprising a sequence selected from SEQ ID NOs: 10-201 in a biological sample from an animal prior to versus after contacting the sample with a test agent, wherein a modulation of expression after contact with the agent relative to prior to contact with the agent is indicative that the agent is capable of (a) inducing the level of memory T cells, (b) promoting the survival of memory T cells, or (c) both (a) and (b).

5. The method of claim 4, wherein said memory T cells are central memory T cells, wherein said modulation is an increase and wherein said one or more nucleic acids or polypeptides comprises a sequence selected from SEQ ID NOs: 10-125 and 198-199.

6. The method of claim 4, wherein said memory T cells are effector memory T cells, wherein said modulation is an increase and wherein said one or more nucleic acids or polypeptides comprises a sequence selected from SEQ ID NOs: 126-197 and 200-201.

7. The method according to claim 4, wherein said method comprises determining the level of expression of at least 2 nucleic acids or polypeptides.

8-11. (canceled)

12. The method of claim 4, wherein said one or more nucleic acids or polypeptides comprises a sequence selected from SEQ ID NOs: 12-25, 38-39, 50-53, 62-63, 82-83, 92-95, 100-107, 110-113, 126-129, 140-151, 154-169 and 174-187.

13. The method of claim 5, wherein said one or more nucleic acids or polypeptides comprises a sequence selected from SEQ ID NOs: 12-25, 38-39, 50-53, 62-63, 82-83, 92-95, 100-107 and 110-113.

14. The method of claim 6, wherein said one or more nucleic acids or polypeptides comprises a sequence selected from SEQ ID NOs: 126-129, 140-151, 154-169 and 174-187.

15. (canceled)

16. A method of identifying an agent capable of inducing protective immunity in an animal, comprising:
(i) providing a first expression profile of one or more nucleic acids or encoding polypeptides selected from BIRC5, CALM1, CAMK2G, CaMKIINalpha, DC-UbP, FAIM2, FOXL2, GATA2, GATA3, IL-7R, IRF1, KIT, MAPK6, MAPKAPK3, RAB11B, STMN1, TNFRSF7 (CD27), CLK1 and PRKARI B in a biological sample from an animal prior to contacting the sample with a test agent;
(ii) providing a second expression profile of one or more nucleic acids encoding a polypeptide selected from BIRC5, CALM1, CAMK2G, CaMKIINalpha, DC-UbP, FAIM2, FOXL2, GATA2, GATA3, IL-7R, IRF1, KIT, MAPK6, MAPKAPK3, RAB11B, STMN1, TNFRSF7 (CD27), CLK1 and PRKARI B in a biological sample from an animal after contacting the sample with the test agent;
(iii) providing a reference expression profile associated with the expression of one or more nucleic acids encoding a polypeptide selected from BIRC5, CALM1, CAMK2G, CaMKIINalpha, DC-UbP, FAIM2, FOXL2, GATA2, GATA3, IL-7R, IRF1, KIT, MAPK6, MAPKAPK3, RAB11B, STMN1, TNFRSF7 (CD27), CLK1 and PRKARI B in a biological sample from an animal exhibiting protective immunity;
wherein increased similarity of the second expression profile to the reference expression profile, relative to the first expression profile to the reference expression profile, is indicative that the agent is capable of inducing protective immunity.

17. A method of identifying an agent capable of inducing protective immunity in an animal, comprising determining the expression of one or more nucleic acids or polypeptides selected from BIRC5, CALM 1, CAM K2G, CaMKIINalpha, DC-UbP, FAIM2, FOXL2, GATA2, GATA3, IL-7R, IRF1, KIT, MAPK6, MAPKAPK3, RAB11B, STMN1, TNFRSF7 (CD27), CLK1 and PRKARI B in a biological sample from an animal prior to versus after contacting the sample with a test agent, wherein a modulation of expression after contact with the agent relative to prior to contact with the agent is indicative that the agent is capable of inducing protective immunity.

18. The method of claim 17, wherein said modulation is an increase and wherein said one or more nucleic acids or polypeptides is selected from BIRC5, CALM1, CAMK2G, CaMKIINalpha, DC-UbP, FAIM2, FOXL2, GATA2, GATA3, IL-7R, IRF1, KIT, MAPK6, MAPKAPK3, RAB11B, STMN1 and TNFRSF7 (CD27).

19. The method of claim 17, wherein said modulation is a decrease and wherein said one or more nucleic acids or encoding polypeptides is selected from CLK1 and PRKARI B.

20. The method according to claim 17, wherein said agent is a vaccine.

21. The method according to claim 16, wherein the subject exhibiting protective immunity is a subject vaccinated with a vaccine known to confer immune protection.

22. The method according to claim 21, wherein said vaccine is Yellow Fever vaccine.

23. The method according to claim 16, wherein said method comprises providing the expression profile of at least 2 nucleic acids or polypeptides.

24-31. (canceled)

32. The method of claim 16, wherein said biological sample comprises central memory T cell (T_{CM}).

33-37. (canceled)

38. A method of inducing the survival of a memory T cell, said method comprising contacting said cell with an agent capable of phosphorylating Foxo3a.

39. A method of increasing immune function in a subject, said method comprising inducing the phosphorylation of Foxo3a in an immune cell of said subject.

40. The method of claim 39, wherein said immune function is memory T cell function.

41. The method of claim 40, wherein said memory T cell function is memory T cell survival.

42. A method of determining whether an HIV-positive subject possesses natural resistance to the development of AIDS, said method comprising:
(i) providing a first expression profile of one or more nucleic acids encoding a polypeptide selected from XIAP, GADD45, DUSP1, PTEN, SOCS1 and SOCS2 in a biological sample from said subject,
(ii) providing a reference expression profile of said one or more nucleic acids in a biological sample from a reference subject known to be an HIV-positive long term non-progressor, wherein a similarity of the first expression profile to the reference expression profile is indicative that the HIV-infected subject possesses natural resistance to the development of AIDS.

43. A collection of two or more isolated nucleic acid sequences which are substantially identical to two or more isolated respective nucleic acid sequences encoding two or more respective polypeptides selected from SEQ ID NOs: 10-201, their complements or portions thereof.

44. The collection of claim 43, comprising at least 5 isolated nucleic acid sequences encoding at least 5 polypeptides, their complements or portions thereof.

45-50. (canceled)

51. The collection of claim 43, wherein said isolated nucleic acid sequences are hybridizable array elements in a microarray.

52-67. (canceled)

68. The method of claim 17, wherein said biological sample comprises a central memory T cell (T_{CM}).
